(19) United States
(12) Patent Application Publication
(10) Pub. No.: US 2021/0036232 A1
(43) Pub. Date: Feb. 4, 2021

(54) ORGANIC COMPOUND, ORGANIC LIGHT-EMITTING DEVICE, DISPLAY DEVICE, PHOTOELECTRIC CONVERSION APPARATUS, ELECTRONIC APPARATUS, LIGHTING DEVICE, AND MOVABLE BODY

(71) Applicant: CANON KABUSHIKI KAISHA, Tokyo (JP)

(72) Inventors: Satoru Shiobara, Hiratsuka-shi (JP); Naoki Yamada, Inagi-shi (JP); Hirokazu Miyashita, Ebina-shi (JP); Yuto Ito, Kokubunji-shi (JP); Isao Kawata, Kawasaki-shi (JP); Jun Kamatani, Tokyo (JP)

(21) Appl. No.: 16/942,554

(22) Filed: Jul. 29, 2020

(30) Foreign Application Priority Data
Aug. 2, 2019 (JP) 2019-143120

(51) Int. Cl. H01L 51/00; C07C 13/62; C09K 11/06

(52) U.S. Cl. CPC H01L 51/0056; C07C 13/62; C09K 11/06; H01L 51/5012; C09K 2211/1007; C09K 2211/1011; C07C 2603/54

(57) ABSTRACT

An organic compound represented by formula [1]:

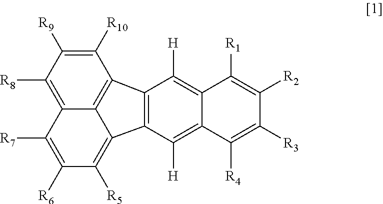

wherein in formula [1], R₁ to R₁₀ are each independently selected from the group consisting of a hydrogen atom, an alkyl group, an alkoxy group, an aryl group, a heterocyclic group, and an amino group, provided that at least one of R₆ and R₉ is a methyl group, and sets of R₁ and R₂, R₂ and R₃, and R₃ and R₄ are each independently optionally taken together to form a ring.

### BACKGROUND OF THE INVENTION

### Field of the Invention

The present disclosure relates to an organic compound, an organic light-emitting device including the organic compound, and a display device, a photoelectric conversion device, electronic apparatus, a lighting device, and a movable body each including the organic light-emitting device.

### Description of the Related Art

In recent years, self-luminous devices usable for flat panel displays have been receiving attention. Examples of self-luminous devices include plasma display devices, field-emission devices, and organic light-emitting devices. Among these, in particular, research and development of organic light-emitting devices has been actively pursued. Expanding the color gamut of a display is a technical issue. Attempts are continuing to develop a device structure for an organic light-emitting device and expand the color gamut by developing a light-emitting material. As color gamuts used for displays, sRGB and Adobe RGB profiles are used. Materials that reproduce them have been sought. BT.2020 nowadays is used as a profile that further expands the color gamut.

To expand the color gamut of an organic light-emitting device, it is known that the optical interference conditions of the device structure can be matched with the peak wavelength of a light-emitting material. However, it is known that it is not easy to find a light-emitting material having a desired emission peak and it is difficult to expand the color gamut. Japanese Patent Laid-Open No. 11-40360 discloses a blue-light-emitting material. Although various substituents on the basic skeleton are exemplified, studies on wavelength control using substituents are not sufficient.

The introduction of a substituent into the molecular structure increases the emission wavelength because of the effect of extended conjugation length and a reduction in molecular symmetry, and thus makes it difficult to obtain a shorter emission wavelength. For example, in the case where the optical interference conditions are matched with the peak wavelength of the blue-light-emitting material by slightly shortening the emission peak wavelength of the light-emitting material, the molecular design of a light-emitting material needs to be redone from the beginning: for example, changing the basic skeleton of the molecular structure. That is, hitherto, there has been no known method for shortening the emission peak wavelength by introducing a substituent into a basic skeleton.

### SUMMARY OF THE INVENTION

The inventors of the present disclosure have conducted studies to solve the above disadvantages and have found that a light-emitting material containing a methyl group introduced at a predetermined position has a basic skeleton exhibiting a shorter emission wavelength and has high sublimation properties. The present disclosure provides an organic compound containing a methyl group introduced at a predetermined position, the organic compound having a shorter emission wavelength and high sublimation properties.

An organic compound according to an embodiment of the present disclosure is represented by formula

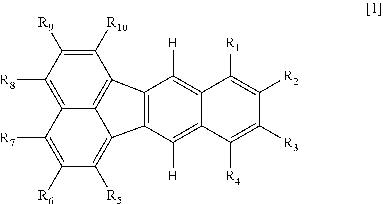

wherein in formula [1], R₁ to R₁₀ are each independently selected from the group consisting of a hydrogen atom, a substituted or unsubstituted alkyl group, a substituted or unsubstituted alkoxy group, a substituted or unsubstituted aryl group, a substituted or unsubstituted heterocyclic group, and a substituted or unsubstituted amino group, provided that at least one of R₆ and R₉ is a methyl group, and sets of R₁ and R₂, R₂ and R₃, and R₃ and R₄ are each independently optionally taken together to form a ring.

Further features of the present disclosure will become apparent from the following description of exemplary embodiments with reference to the attached drawings.

### BRIEF DESCRIPTION OF THE DRAWINGS

FIG. 1 is a conceptual diagram explaining the shifts of HOMO and LUMO levels when a substituent is introduced into a basic skeleton.

FIG. 2 is a schematic cross-sectional view of an example of a display device including organic light-emitting devices according to an embodiment of the present disclosure.

FIG. 3 is a schematic view illustrating an example of a display device according to an embodiment of the present disclosure.

FIG. 4A is a schematic view illustrating an example of an image pickup apparatus according to an embodiment of the present disclosure, and FIG. 4B is a schematic view illustrating an example of an electronic apparatus according to an embodiment of the present disclosure.

FIG. 5A is a schematic view illustrating an example of a display device according to an embodiment of the present disclosure, and FIG. 5B is a schematic view illustrating an example of a foldable display device.

FIG. 6A is a schematic view illustrating an example of a lighting device according to an embodiment of the present disclosure, and FIG. 6B is a schematic view illustrating an example of an automobile including a lighting tool for a vehicle according to an embodiment of the present disclosure.

### DESCRIPTION OF THE EMBODIMENTS

Organic Compound

An organic compound according to an embodiment will first be described. The organic compound according to the embodiment is represented by formula [1]:

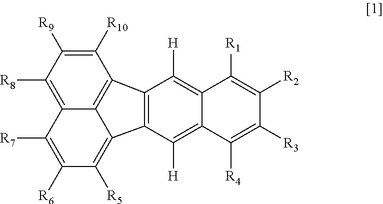

wherein in formula [1], R₁ to R₁₀ are each independently selected from the group consisting of a hydrogen atom, a substituted or unsubstituted alkyl group, a substituted or unsubstituted alkoxy group, a substituted or unsubstituted aryl group, a substituted or unsubstituted heterocyclic group, and a substituted or unsubstituted amino group, provided that at least one of R₆ and R₉ is a methyl group, and sets of R₁ and R₂, R₂ and R₃, and R₃ and R₄ are each independently optionally taken together to form a ring.

The basic skeleton used in this specification refers to the largest condensed ring structure in its molecule, the structure determining a fundamental emission wavelength range. Specifically, the basic skeleton is a skeleton in which R₁ to R₁₀ of the compound represented by formula [1] are all hydrogen atoms. However, in the case where at least one of the sets of R₁ and R₂, R₂ and R₃, and R₃ and R₄ is taken together to form a ring, the basic skeleton refers to a skeleton in which R₅ to R₁₀ and a group that does not form a ring out of R₁ to R₄ are all hydrogen atoms and in which the formed ring is unsubstituted (a ring consisting only of ring atoms and hydrogen atoms).

R₁ to R₁₀ can each be independently selected from a hydrogen atom, a substituted or unsubstituted alkyl group, and a substituted or unsubstituted aryl group, even a hydrogen atom and a substituted or unsubstituted aryl group.

Each of R₅, R₇, R₈, and R₁₀ can be a hydrogen atom, an aryl group, or a heterocyclic group bonded to an atom of the basic skeleton through a carbon atom. A group that does not form a ring out of R₁ to R₄ can be a hydrogen atom, an aryl group, or a heterocyclic group bonded to an atom of the basic skeleton through a carbon atom. Each of R₆ and R₉ can be a methyl group. In the case where only one of R₆ and R₉ is a methyl group, the other can be a hydrogen atom.

Examples of alkyl groups denoted by R₁ to R₁₀ include, but are not limited to, a methyl group, an ethyl group, a normal propyl group, an isopropyl group, a normal butyl group, a tertiary butyl group, a secondary butyl group, an octyl group, a cyclohexyl group, a 1-adamantyl group, and a 2-adamantyl group. An alkyl group denoted by each of R₁ to R₁₀ can be an alkyl group having 1 or more and 10 or less carbon atoms.

Examples of alkoxy groups denoted by R₁ to R₁₀ include, but are not limited to, a methoxy group, an ethoxy group, a propoxy group, a 2-ethylhexyloxy group, and a benzyloxy group.

Examples of aryl groups denoted by R₁ to R₁₀ include, but are not limited to, a phenyl group, a naphthyl group, an indenyl group, a biphenyl group, a terphenyl group, and a fluorenyl group. An aryl group denoted by each of R₁ to R₁₀ can be an aryl group having 6 or more and 18 or less carbon atoms.

Examples of heterocyclic groups denoted by R₁ to R₁₀ include, but are not limited to, a pyridyl group, an oxazolyl group, an oxadiazolyl group, a thiazolyl group, a thiadiazolyl group, a carbazolyl group, an acridinyl group, and a phenanthryl group. A heterocyclic group denoted by each of R₁ to R₁₀ can be a heterocyclic group bonded to an atom of the basic skeleton through a carbon atom.

Examples of amino groups denoted by R₁ to R₁₀ include, but are not limited to, an N-methylamino group, an N-ethylamino group, an N,N-dimethylamino group, an N,N-diethyl amino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-tert-butylphenyl)amino group, an N-phenyl-N-(4-trifluoromethylphenyl)amino group, and an N-piperidyl group.

Examples of substituents optionally contained in the alkyl groups, the alkoxy groups, the aryl groups, the heterocyclic groups, and the amino groups described above include, but are not limited to, alkyl groups, such as a methyl group, an ethyl group, a normal propyl group, an isopropyl group, a normal butyl group, and a tertiary butyl group; aralkyl groups, such as a benzyl group; aryl groups, such as a phenyl group and a biphenyl group; heterocyclic groups, such as a pyridyl group and a pyrrolyl group; amino groups, such as a dimethylamino group, a diethylamino group, a dibenzylamino group, a diphenylamino group, and a ditolylamino group; alkoxy groups, such as a methoxy group, an ethoxy group, and a propoxy group; and aryloxy groups, such as a phenoxy group. Among these, alkyl groups, aralkyl groups, and aryl groups can be used.

At least one of sets of R₁ and R₂, R₂ and R₃, and R₃ and R₄ can be taken together to form a ring. The ring formed may be a single or condensed ring. The ring formed may have a substituent. Examples of the substituent optionally contained in the ring formed include a hydrogen atom, a substituted or unsubstituted alkyl group, a substituted or unsubstituted alkoxy group, a substituted or unsubstituted aryl group, a substituted or unsubstituted heterocyclic group, and a substituted or unsubstituted amino group. The substituent can be a substituted or unsubstituted aryl group or a heterocyclic group bonded to an atom of the basic skeleton through a carbon atom. The ring formed can be an aromatic ring, such as a condensed polycyclic aromatic ring, even a condensed polycyclic aromatic ring including a five-membered carbon ring.

In the case where at least one of the sets of R₁ and R₂, R₂ and R₃, and R₃ and R₄ is taken together to form a ring, examples of the resulting structure are illustrated below. In each of G1 to G15, R₅, R₇, R₈, and R₁₀ are each a hydrogen atom, and R₆ and R₉ are each a methyl group. However, these groups are not limited thereto. In each of G1 to G3, G6 to G9, and G11 to G15, a group that does not form a ring out of R₁ to R₄ is a substituted or unsubstituted phenyl group but is not limited thereto.

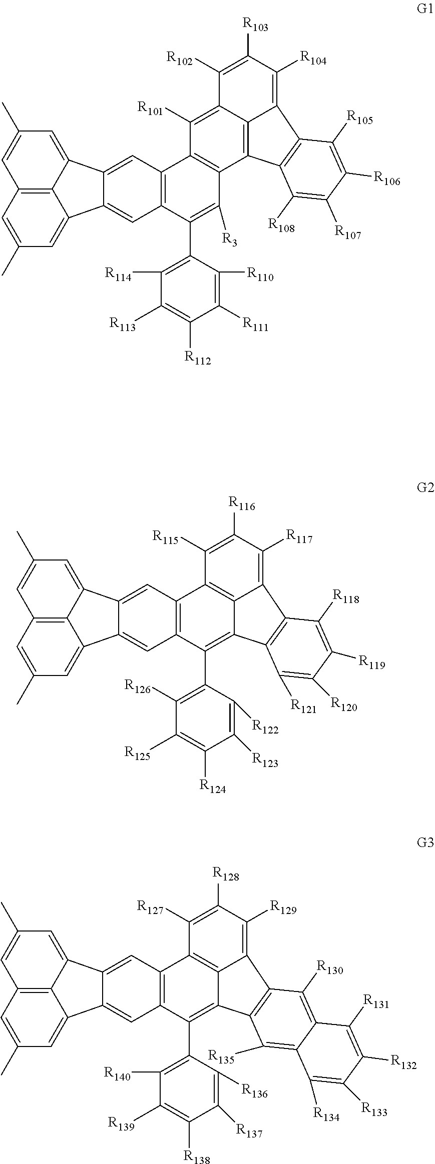

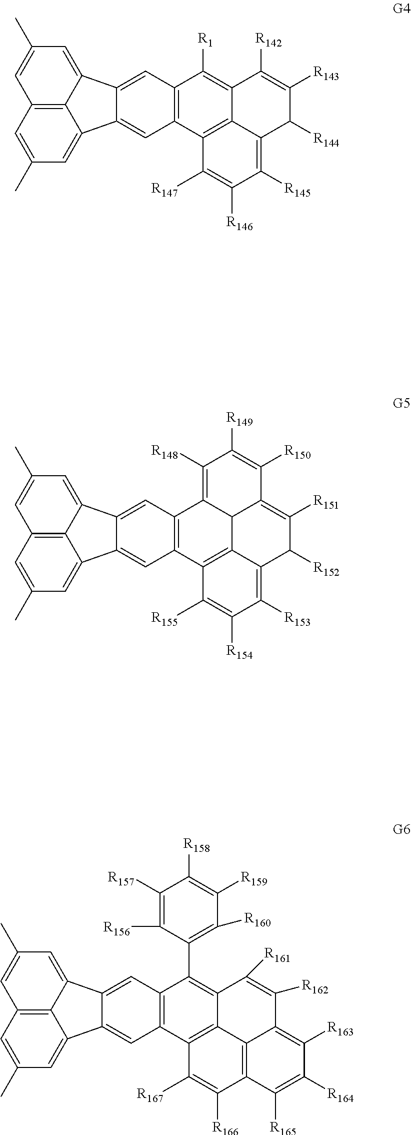

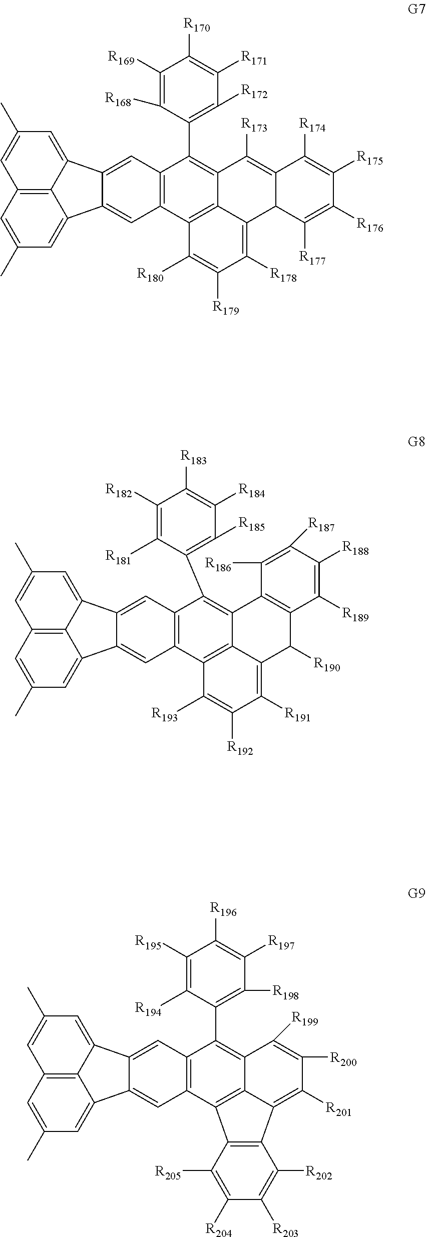

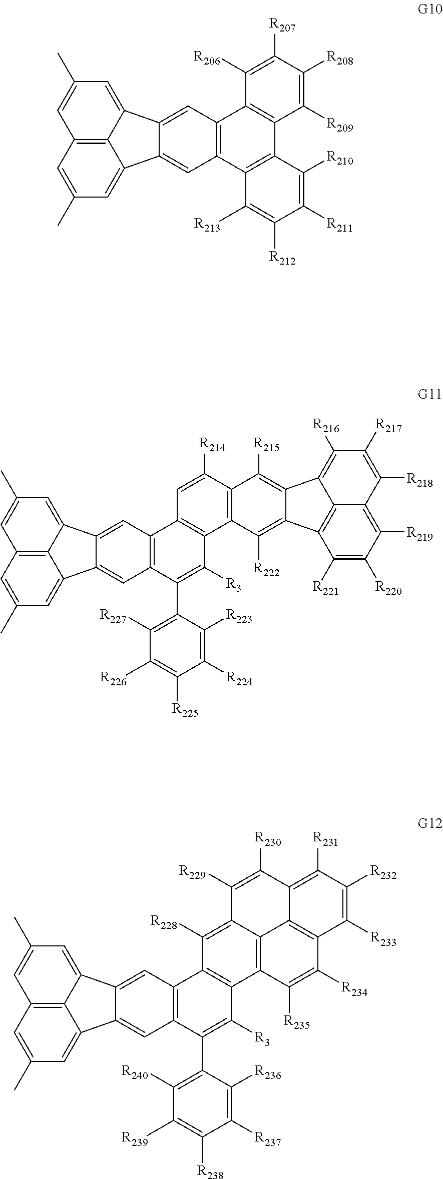

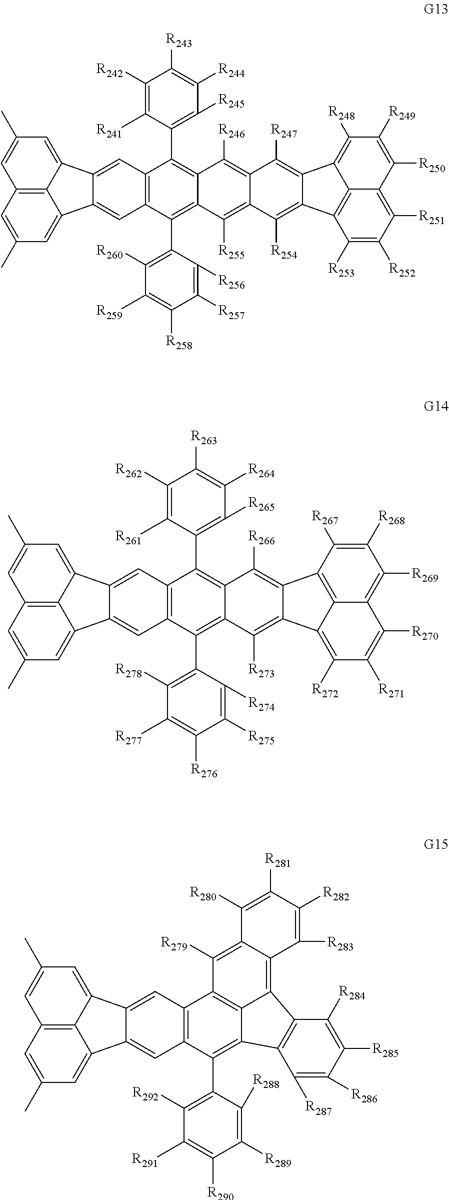

In each of G1 to G15, R₁₀₁ to R₁₀₈, R₁₁₀ to R₁₄₀, and R₁₄₂ to R₂₉₂ are each a hydrogen atom, a substituted or unsubstituted alkyl group, a substituted or unsubstituted alkoxy group, a substituted or unsubstituted aryl group, a substituted or unsubstituted heterocyclic group, or a substituted or unsubstituted amino group.

R₁₀₁ to R₁₀₈, R₁₁₀ to R₁₄₀, and R₁₄₂ to R₂₉₂ can each be independently selected from a hydrogen atom, a substituted or unsubstituted alkyl group, and a substituted or unsubstituted aryl group, even a hydrogen atom and a substituted or unsubstituted aryl group.

Examples of alkyl groups denoted by R₁₀₁ to R₁₀₈, R₁₁₀ to R₁₄₀, and R₁₄₂ to R₂₉₂ include, but are not limited to, a methyl group, an ethyl group, a normal propyl group, an isopropyl group, a normal butyl group, a tertiary butyl group, a secondary butyl group, an octyl group, a cyclohexyl group, a 1-adamantyl group, and a 2-adamantyl group. The alkyl groups denoted by R₁₀₁ to R₁₀₈, R₁₁₀ to R₁₄₀, and R₁₄₂ to R₂₉₂ can each be an alkyl group having 1 or more and 10 or less carbon atoms.

Examples of alkoxy groups denoted by R₁₀₁ to R₁₀₈, R₁₁₀ to R₁₄₀, and R₁₄₂ to R₂₉₂ include, but are not limited to, a methoxy group, an ethoxy group, a propoxy group, a 2-ethylhexyloxy group, and a benzyloxy group.

Examples of aryl groups denoted by R₁₀₁ to R₁₀₈, R₁₁₀ to R₁₄₀, and R₁₄₂ to R₂₉₂ include, but are not limited to, a phenyl group, a naphthyl group, an indenyl group, a biphenyl group, a terphenyl group, and a fluorenyl group. The aryl groups denoted by R₁₀₁ to R₁₀₈, R₁₁₀ to R₁₄₀, and R₁₄₂ to R₂₉₂ can each be an aryl group having 6 or more and 18 or less carbon atoms.

Examples of heterocyclic groups denoted by R₁₀₁ to R₁₀₈, R₁₁₀ to R₁₄₀, and R₁₄₂ to R₂₉₂ include, but are not limited to, a pyridyl group, an oxazolyl group, an oxadiazolyl group, a thiazolyl group, a thiadiazolyl group, a carbazolyl group, an acridinyl group, and a phenanthryl group. The heterocyclic groups denoted by R₁₀₁ to R₁₀₈, R₁₁₀ to R₁₄₀, and R₁₄₂ to R₂₉₂₀ can each be a heterocyclic group bonded to an atom of the basic skeleton through a carbon atom.

Examples of amino groups denoted by R₁₀₁ to R₁₀₈, R₁₁₀ to R₁₄₀, and R₁₄₂ to R₂₉₂ include, but are not limited to, an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, a dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-tert-butylphenyl)amino group, an N-phenyl-N-(4-trifluoromethylphenyl)amino group, and an N-piperidyl group.

Examples of substituents optionally contained in the alkyl groups, the alkoxy groups, the aryl groups, the heterocyclic groups, and the amino groups described above include, but are not limited to, alkyl groups, such as a methyl group, an ethyl group, a normal propyl group, an isopropyl group, a normal butyl group, and a tertiary butyl group; aralkyl groups, such as a benzyl group; aryl groups, such as a phenyl group and a biphenyl group; heterocyclic groups, such as a pyridyl group and a pyrrolyl group; amino groups, such as a dimethylamino group, a diethylamino group, a dibenzylamino group, a diphenylamino group, and a ditolylamino group; alkoxy groups, such as a methoxy group, an ethoxy group, and a propoxy group; and aryloxy groups, such as a phenoxy group. Among these, alkyl groups, aralkyl groups, and aryl groups can be used.

The organic compound according to the embodiment has the following characteristics and thus can have a shorter emission wavelength. Thus, an organic light-emitting device can have improved chromaticity coordinates and an expanded color gamut. The organic light-emitting device containing the organic compound according to the embodiment has excellent durability characteristics.

(1) Since at least one of R₆ and R₉ is a methyl group, a short emission wavelength is obtained. (2) Since at least one of R₆ and R₉ is a methyl group, the organic compound is not easily decomposed during vacuum evaporation.

Item (1) will be described below. In the organic compound according to the embodiment, at least one of R₆ and R₉ in formula [1] is a methyl group. For the sake of clarity, the explanation will be made in comparison with the case where R₆ and R₉ are each a hydrogen atom. The introduction of a methyl group into at least one of R₆ and R₉ changes HOMO and LUMO levels of the basic skeleton because of the interaction between the basic skeleton and the substituent. For example, when a methyl group is introduced as a substituent, the HOMO and LUMO levels shift toward the vacuum level in the direction of lower ionization potential energy because a methyl group has an electron-donating ability. The change in level is referred to as a “shift amount”.

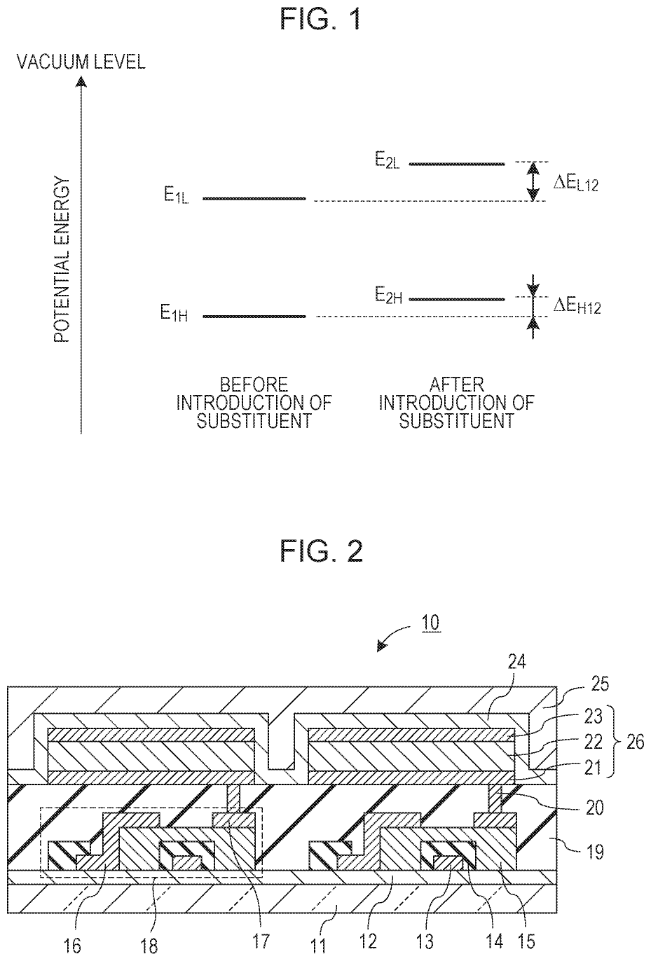

To shorten the emission wavelength by introducing a methyl group, the shift amount of the LUMO level needs to be larger than the shift amount of the HOMO level when a methyl group is introduced. This will be described with reference to FIG. 1. In FIG. 1, regarding the basic skeleton before the introduction of the substituent, the HOMO potential energy level is denoted by E_{1H}, and the LUMO potential energy level is denoted by E_{1L}. Regarding a compound obtained by introducing the substituent into the basic skeleton, the HOMO potential energy level is denoted by E_{2H}, and the LUMO potential energy level is denoted by E_{2L}. The shift amounts of HOMO and LUMO levels due to the introduction of the substituent into the basic skeleton are ΔE_{H12} and ΔE_{L12}, respectively. In the case where ΔE_{H12}<ΔE_{L12}, the introduction of the methyl group increases the band gap to shorten the emission wavelength.

Usually, the introduction of a methyl group greatly increases the HOMO level to decrease the band gap, thereby increasing the emission wavelength. To shorten the emission wavelength, it is important to attach a substituent to a specific position. The inventors have conducted intensive studies on the substitution position and have found that in formula [1], a compound in which R₆ and R₉ are each a methyl group had a shorter emission wavelength than a compound in which R₆ and R₉ are each a hydrogen atom.

The inventors have examined the reason for a decrease in emission wavelength due to the substitution of the methyl groups at the positions and have found that the emission wavelength can be decreased by substitution at the 6- and 9-positions of a fluoranthene structure. In contrast, the emission wavelength was not decreased at any other binding position.

An emission wavelength region where this effect is particularly effective is not particularly limited. In the case of the blue region with high color purity, however, the maximum emission wavelength in a dilute toluene solution is in the range of 420 nm to 480 nm. The reason for this is that in the case of blue light, a decrease in the wavelength of an emission spectrum contributes to the expansion of the color gamut.

The reason for item (2) will be described below. The effect of the methyl group described in item (1) is common to electron-donating substituents. In the case of introducing a substituent that does not contain a hetero bond, a methyl group can be used because the methyl group serves as a substituent having a minimum molecular weight among substituents capable of imparting an electron-donating property. The reason why the methyl group can be used is that the methyl group is not easily decomposed or is not decomposed during sublimation. Let us take a tert-butyl group as an example. The introduction of a tert-butyl group increases the intermolecular distance and the intermolecular force because of a large increase in molecular weight. This increases energy required to separate molecules from each other during sublimation to decompose the tert-butyl group. The decomposition products are incorporated in a film during film formation; thus, the operating life is shortened.

Regarding compounds that differ in R₆ and R₉ of formula [1], a degradation in the degree of vacuum during vacuum evaporation was evaluated by the following method. Specifically, the degradation in the degree of vacuum was evaluated when the following compounds were used: experiment A: exemplified compound A17 illustrated below; experiment B: a compound in which R₆ and R₉ of exemplified compound A17 were changed to ethyl groups; experiment C: a compound in which R₆ and R₉ of exemplified compound A17 were changed to tert-butyl groups; experiment D: exemplified compound A22 illustrated below; experiment E: a compound in which R₆ and R₉ of exemplified compound A22 were changed to ethyl groups; and experiment F: a compound in which R₆ and R₉ of exemplified compound A22 were changed to tert-butyl groups. Table 1 presents the results.

(Vapor Deposition Test)

a) First, 30 mg of each compound was placed on a Mo boat for resistance heating. The boat was placed in a vacuum evaporation apparatus (VPC-1100). The apparatus was evacuated to a pressure of 4×10⁻⁵ Pa.
b) After performing the operations described in item a), resistance heating was performed while the thickness was monitored with CRTM 9000. The degree of vacuum when the deposition rate reached 0.5 Å/s was compared with the degree of vacuum before resistance heating to evaluate the presence or absence of the degradation in the degree of vacuum.

TABLE 1

| | | | |
|---|---|---|---|
| | Experiment A | Experiment B | Experiment C |
| Molecular structure | 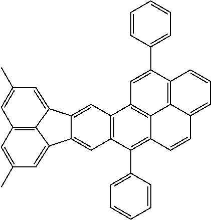 [SMILES: Cc1cc2c3c(cc(C)cc3c1)-c1cc3c(cc1-2)c(-c1ccccc1)c1ccc2cccc4c(-c5ccccc5)cc3c1c24] | 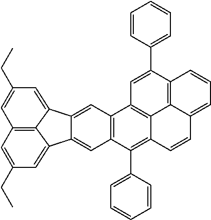 [SMILES: CCc1cc2c3c(cc(CC)cc3c1)-c1cc3c(cc1-2)c(-c1ccccc1)c1ccc2cccc4c(-c5ccccc5)cc3c1c24] | 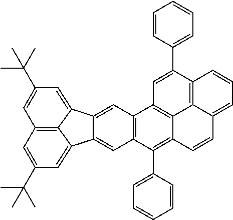 [SMILES: CC(C)(C)c1cc2c3c(cc(C(C)(C)C)cc3c1)-c1cc3c(cc1-2)c(-c1ccccc1)c1ccc2cccc4c(-c5ccccc5)cc3c1c24] |
| Decrease in | no | yes | yes |
| degree of | | | |
| vacuum | | | |
| at start of | | | |
| vacuum | | | |
| deposition | | | |
| | Experiment D | Experiment E | Experiment F |
| Molecular structure | 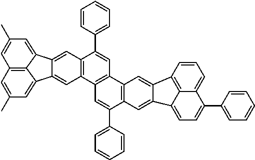 [SMILES: Cc1cc2c3c(cc(C)cc3c1)-c1cc3c(cc1-2)c(-c1ccccc1)cc1c2cc4c(cc2c(-c2ccccc2)cc31)-c1ccc(-c2ccccc2)c2cccc-4c12] | 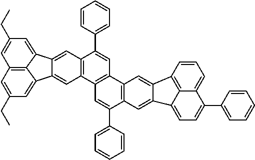 [SMILES: CCc1cc2c3c(cc(CC)cc3c1)-c1cc3c(cc1-2)c(-c1ccccc1)cc1c2cc4c(cc2c(-c2ccccc2)cc31)-c1ccc(-c2ccccc2)c2cccc-4c12] | 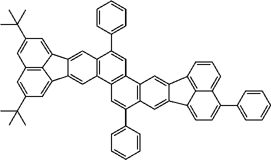 [SMILES: CC(C)(C)c1cc2c3c(cc(C(C)(C)C)cc3c1)-c1cc3c(cc1-2)c(-c1ccccc1)cc1c2cc4c(cc2c(-c2ccccc2)cc31)-c1ccc(-c2ccccc2)c2cccc-4c12] |
| Decrease in | no | yes | yes |
| degree of | | | |
| vacuum | | | |
| at start of | | | |
| vacuum | | | |
| deposition | | | |

As presented in Table 1, the methyl groups can be used rather than the tert-butyl groups. The methyl groups can also be used rather than the ethyl groups. The degradation in the degree of vacuum indicates the generation of decomposition products. The decomposition products are incorporated into the films during film formation to cause the durability characteristics of the organic light-emitting devices to deteriorate.

Furthermore, items (3) and (4) described below can be satisfied. (3) Two or more five-membered carbon ring structures can be included. (4) The bond between the basic skeleton and any group other than a hydrogen atom can be a carbon-carbon bond.

Item (3) will be described below. Two or more five-membered ring structures composed of carbon atoms can be included. For example, as illustrated in formula [2] below, two or more moieties each including a fluoranthene structure serving as a molecular skeleton including a five-membered ring can be included.

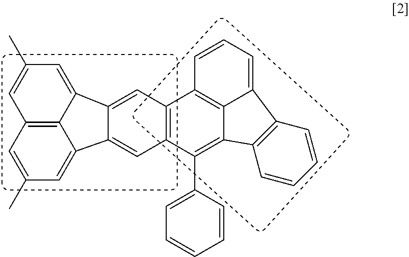 [SMILES: Cc1cc2cc(C)cc3c4cc5c(cc4c(c1)c23)c(-c1ccccc1)c1c2ccccc2c2cccc5c21]

A compound having two five-membered carbon ring structures has a deeper ionization potential than a compound having one five-membered carbon ring structure. The organic compound having a deeper ionization potential has enhanced resistance to oxidation and thus has improved durability characteristics.

Next, item (4) will be described. In the case where none of the sets of R₁ and R₂, R₂ and R₃, and R₃ and R₄ form a ring, R₁ to R₁₀ can each be a hydrogen atom or a group bonded to an atom of the basic skeleton through a carbon atom. In the case where one of the sets of R₁ and R₂, R₂ and R₃, and R₃ and R₄ forms a ring, R₅ to R₁₀, a group that does not form a ring out of R₁ to R₄, and a group bonded to an atom of the formed ring can each be a hydrogen atom or a group bonded to an atom of the basic skeleton through a carbon atom. Examples of the group bonded to an atom of the basic skeleton through a carbon atom include alkyl groups, aryl groups, and heterocyclic groups each bonded through a carbon atom. In particular, the formed ring can be an aromatic ring, and the substituent of the aromatic ring can be an aryl group or a heterocyclic group bonded through a carbon atom. This is because a carbon-carbon bond is the strongest bond with respect to other bonds and thus the deterioration is slowest in terms of operation durability characteristics. The energy of each bond is illustrated below.

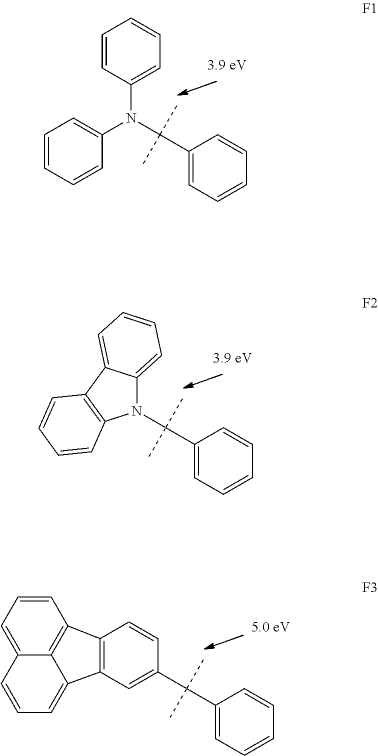

In the case where such a compound is used for an organic light-emitting device, excitons are continuously generated in high density in a light-emitting layer, and light is emitted. Thus, the organic compound is required to have strength enough to withstand endless cycles of excitation and emission. In this respect, a carbon atom-carbon atom bond portion has larger bond energy than a hetero atom-carbon atom bond portion and thus is less likely to be cleaved, and the material is less likely to deteriorate in an excited state during repeated photoexcitation and emission cycles.

Specific examples of the organic compound according to the embodiment will be illustrated below. However, the present invention is not limited to these examples.

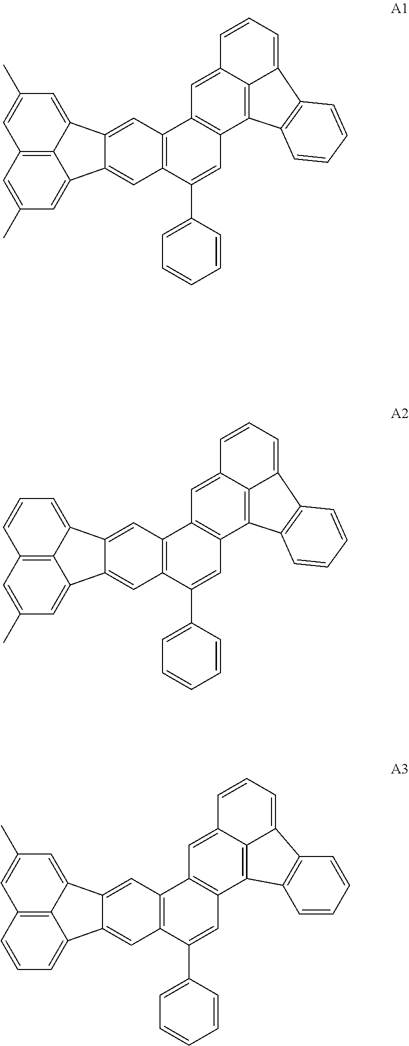

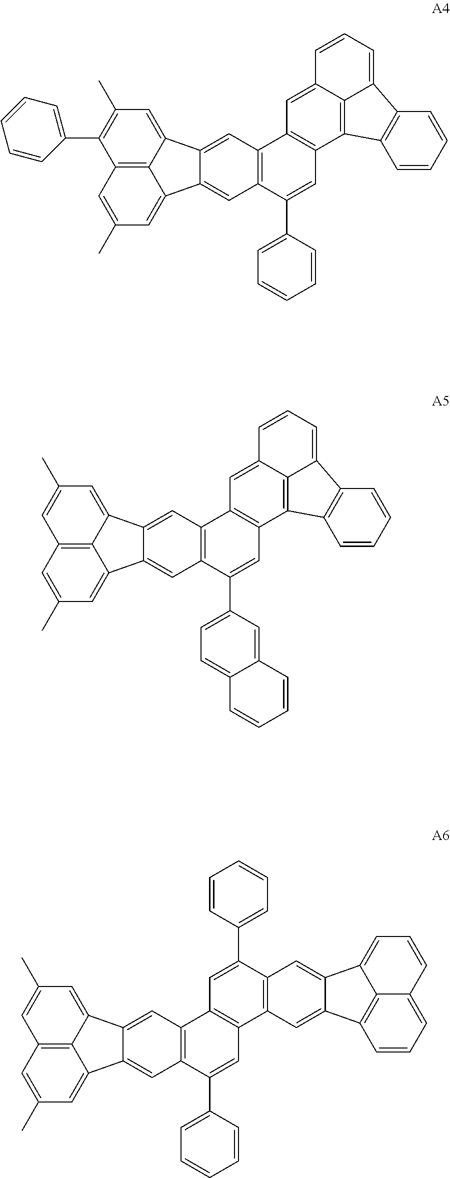

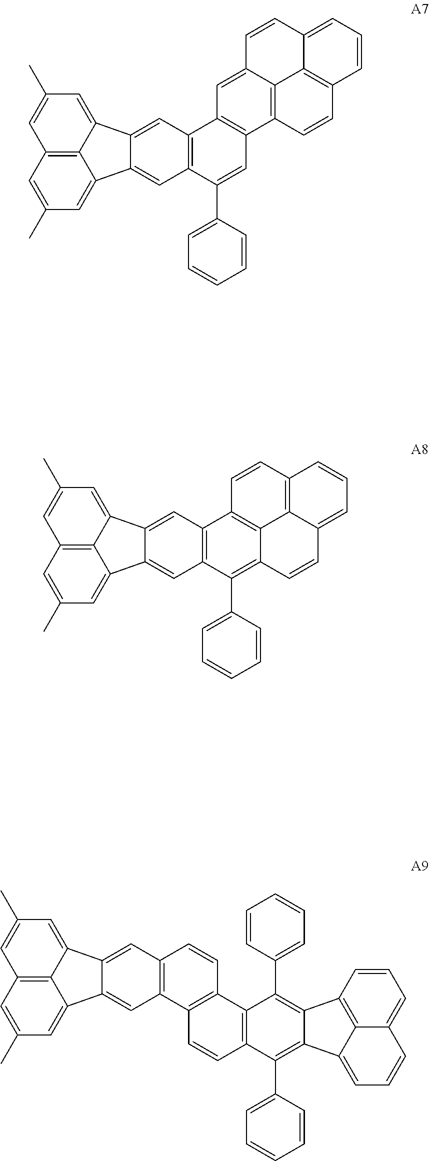

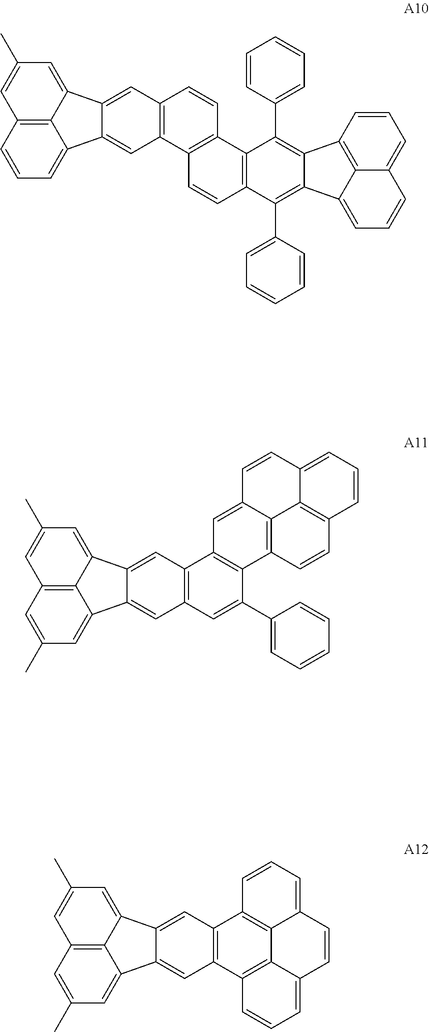

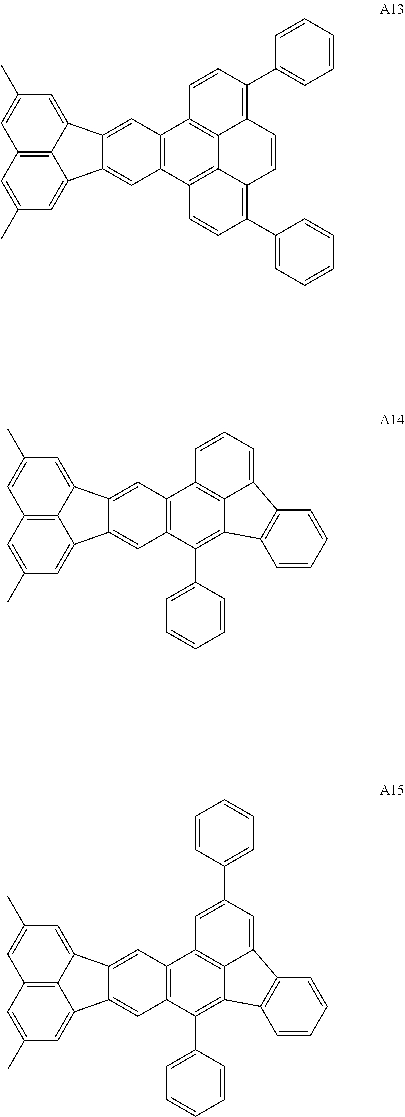

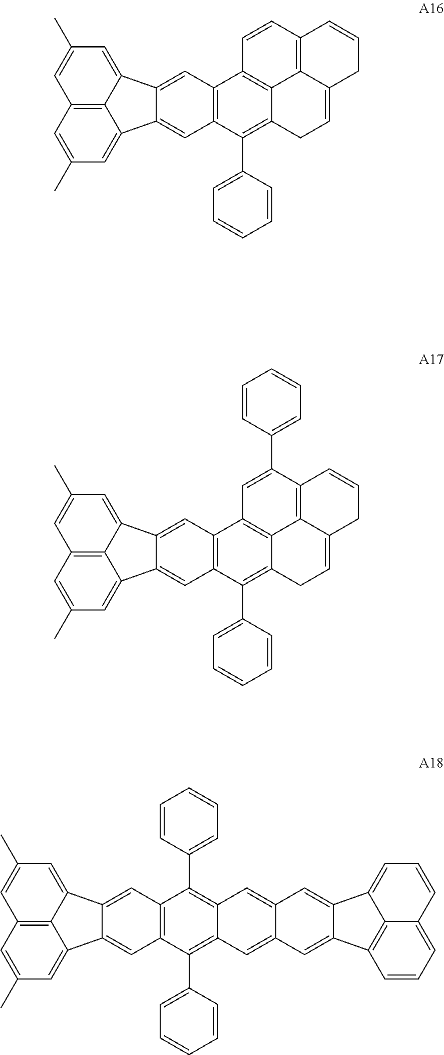

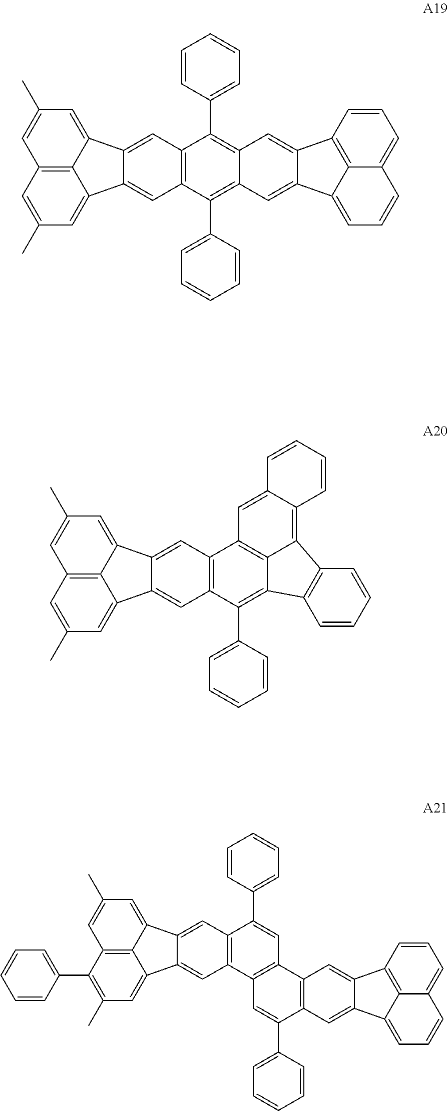

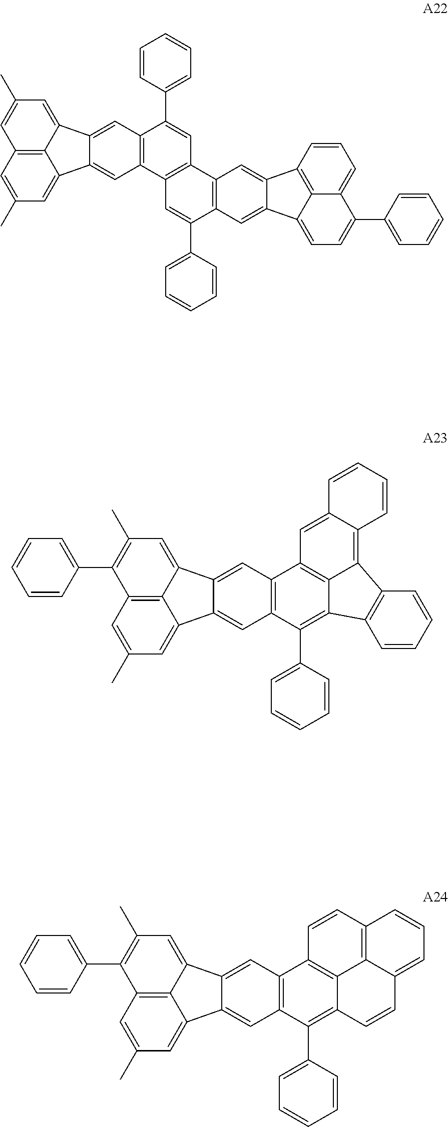

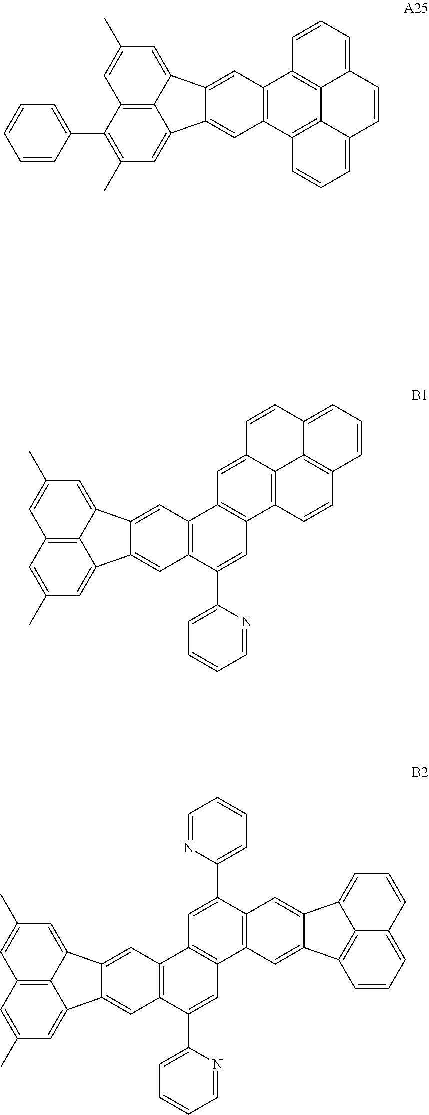

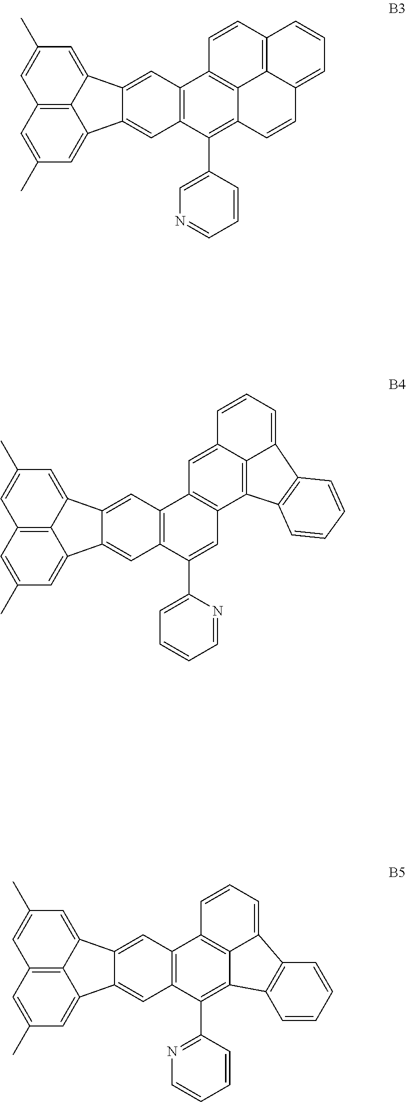 [SMILES: Cc1cc2c3c(cc(C)cc3c1)-c1cc3c(cc1-2)c(-c1cccnc1)c1ccc2cccc4ccc3c1c24 ; Cc1cc2cc(C)cc3c4cc5c(cc4c(c1)c23)c(-c1ccccn1)cc1c5cc2cccc3c4ccccc4c1c23 ; Cc1cc2cc(C)cc3c4cc5c(cc4c(c1)c23)c(-c1ccccn1)c1c2ccccc2c2cccc5c21]

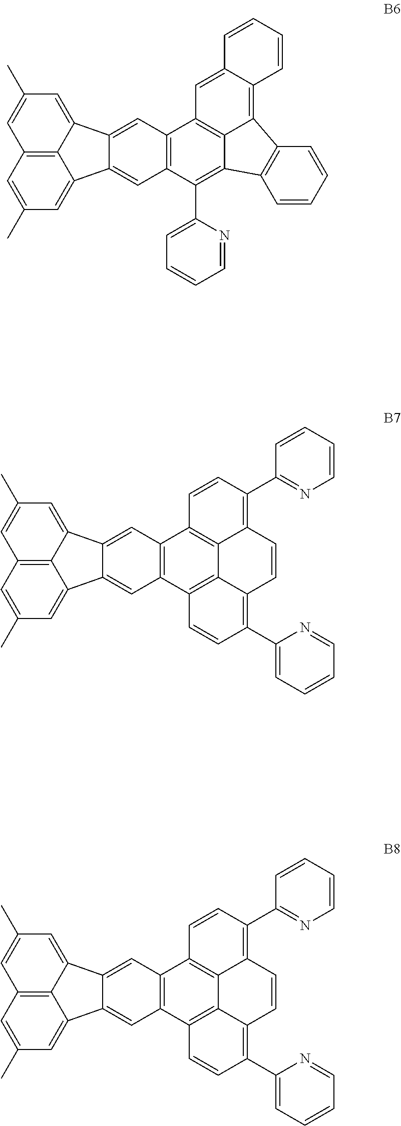 [SMILES: Cc1cc2cc(C)cc3c4cc5c(cc4c(c1)c23)c(-c1ccccn1)c1c2ccccc2c2c3ccccc3cc5c12 ; Cc1cc2c3c(cc(C)cc3c1)-c1cc3c(cc1-2)c1ccc(-c2ccccn2)c2ccc4c(-c5ccccn5)ccc3c4c21 ; Cc1cc2c3c(cc(C)cc3c1)-c1cc3c(cc1-2)c1ccc(-c2ccccn2)c2ccc4c(-c5ccccn5)ccc3c4c21]

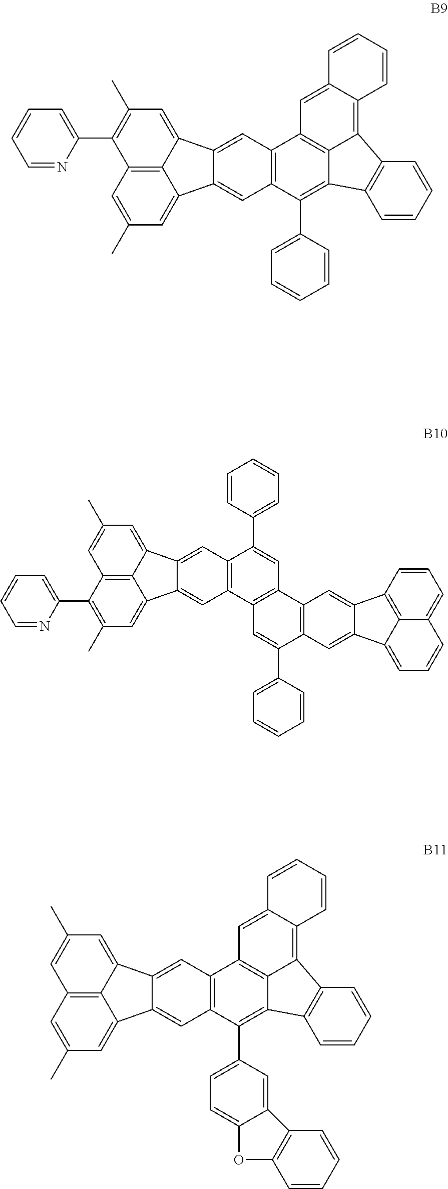 [SMILES: Cc1cc2c(-c3ccccn3)c(C)cc3c4cc5c(cc4c(c1)c23)c(-c1ccccc1)c1c2ccccc2c2c3ccccc3cc5c12 ; Cc1cc2c3c(cc(C)c(-c4ccccn4)c3c1)-c1cc3c(cc1-2)c(-c1ccccc1)cc1c2cc4c(cc2c(-c2ccccc2)cc31)-c1cccc2cccc-4c12 ; Cc1cc2cc(C)cc3c4cc5c(cc4c(c1)c23)c(-c1ccc2oc3ccccc3c2c1)c1c2ccccc2c2c3ccccc3cc5c12]

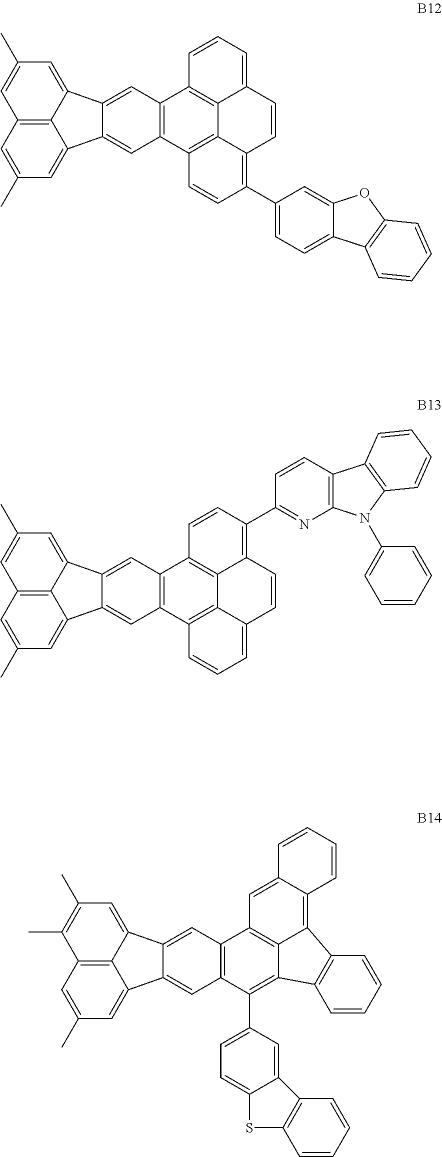 [SMILES: Cc1cc2c3c(cc(C)cc3c1)-c1cc3c(cc1-2)c1cccc2ccc4c(-c5ccc6c(c5)oc5ccccc56)ccc3c4c21 ; Cc1cc2c3c(cc(C)cc3c1)-c1cc3c(cc1-2)c1cccc2ccc4c(-c5ccc6c7ccccc7n(-c7ccccc7)c6n5)ccc3c4c21 ; Cc1cc2c(C)c(C)cc3c4cc5c(cc4c(c1)c23)c(-c1ccc2sc3ccccc3c2c1)c1c2ccccc2c2c3ccccc3cc5c12]

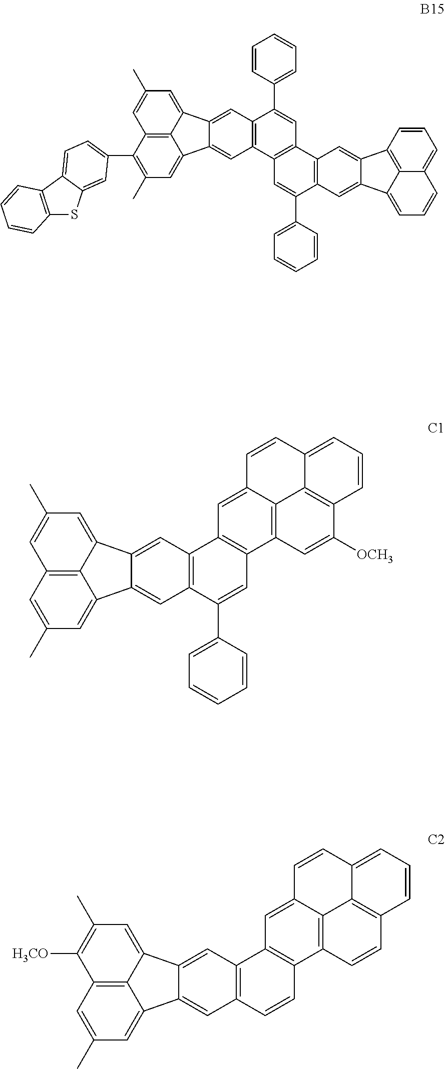 [SMILES: Cc1cc2c3c(cc(C)c(-c4ccc5c(c4)sc4ccccc45)c3c1)-c1cc3c(cc1-2)c(-c1ccccc1)cc1c2cc4c(cc2c(-c2ccccc2)cc31)-c1cccc2cccc-4c12 ; COc1cc2c3cc(-c4ccccc4)c4cc5c(cc4c3cc3ccc4cccc1c4c32)-c1cc(C)cc2cc(C)cc-5c12 ; COc1c(C)cc2c3c(cc(C)cc13)-c1cc3ccc4c(cc5ccc6cccc7ccc4c5c67)c3cc1-2]

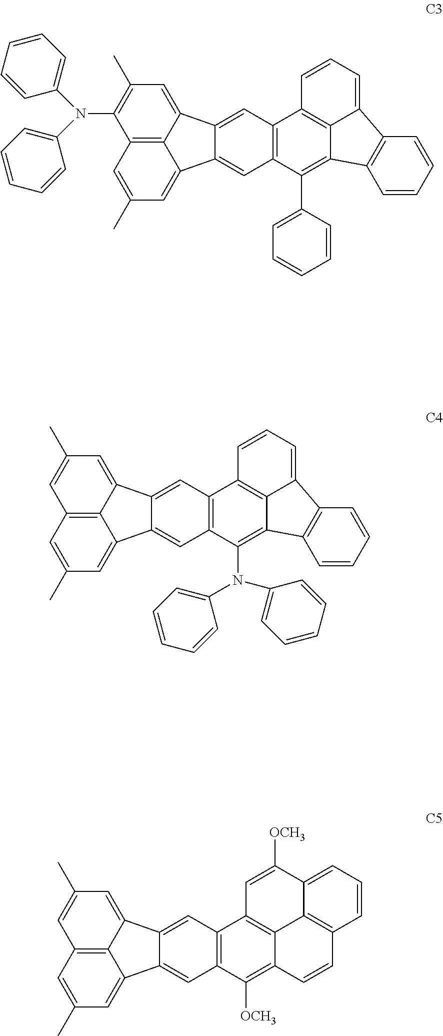 [SMILES: Cc1cc2c(N(c3ccccc3)c3ccccc3)c(C)cc3c4cc5c(cc4c(c1)c23)c(-c1ccccc1)c1c2ccccc2c2cccc5c21 ; Cc1cc2cc(C)cc3c4cc5c(cc4c(c1)c23)c(N(c1ccccc1)c1ccccc1)c1c2ccccc2c2cccc5c21 ; COc1cc2c3cc4c(cc3c(OC)c3ccc5cccc1c5c32)-c1cc(C)cc2cc(C)cc-4c12]

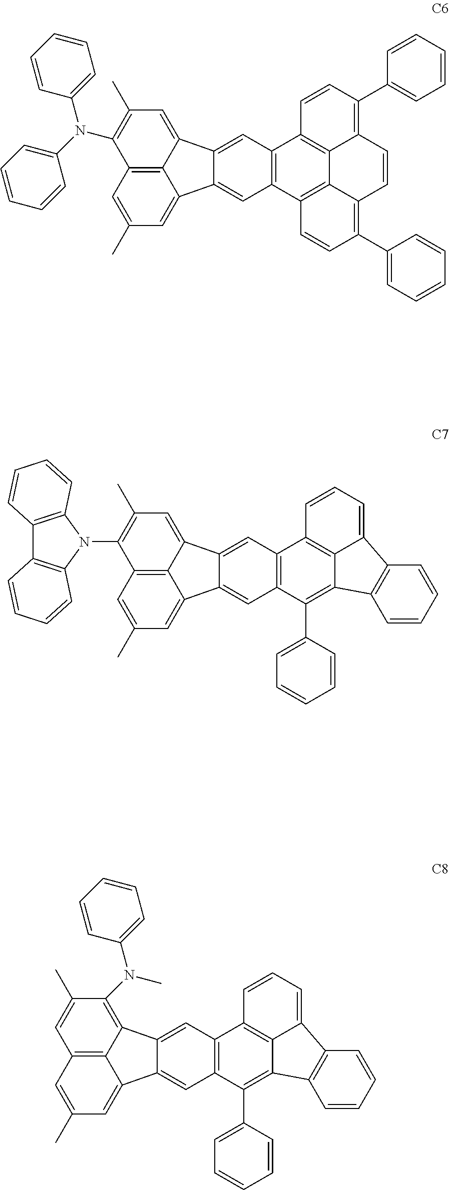 [SMILES: Cc1cc2c3c(cc(C)c(N(c4ccccc4)c4ccccc4)c3c1)-c1cc3c(cc1-2)c1ccc(-c2ccccc2)c2ccc4c(-c5ccccc5)ccc3c4c21 ; Cc1cc2c(-n3c4ccccc4c4ccccc43)c(C)cc3c4cc5c(cc4c(c1)c23)c(-c1ccccc1)c1c2ccccc2c2cccc5c21 ; Cc1cc2cc(C)c(N(C)c3ccccc3)c3c4cc5c(cc4c(c1)c23)c(-c1ccccc1)c1c2ccccc2c2cccc5c21]

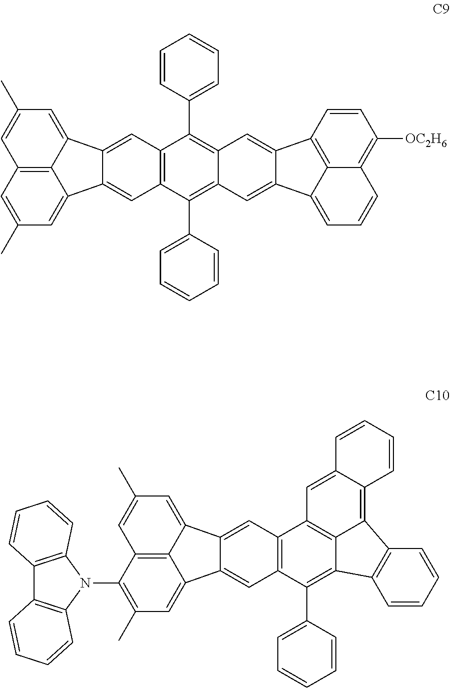

The above exemplified compounds are classified into three groups of A, B, and C. In each case, the compound groups have the structure illustrated in formula [1], and each compound in which at least one of R₆ and R₉ is a methyl group exhibits a shorter emission wavelength. Among groups A to C, group A can have a long operating life.

Group A satisfies (A) to (E) and is a group of compounds whose molecules each consist entirely of carbon and hydrogen.

(A) At least one of the sets of R₁ and R₂, R₂ and R₃, and R₃ and R₄ is taken together to form a ring.

(B) Regarding R₆ and R₉, both are methyl groups, or one of them is a methyl group, and the other is a hydrogen atom.

(C) A group that does not form a ring out of R₁ to R₄ is a hydrogen atom or an aryl group.

(D) R₅, R₇, R₈, and R₁₀ are each a hydrogen atom or an aryl group.

(E) A substituent on an aromatic ring formed by taking at least one of the sets of R₁ and R₂, R₂ and R₃, and R₃ and R₄ together is an aryl group.

Here, compounds each consisting of carbon and hydrogen usually have high ionization potentials. Thus, the compounds belonging to group A are stable to oxidation. Among compounds according to the embodiment, the compounds belonging to group A can have high stability as molecules. The compounds of group A can be used as light-emitting materials and, in addition, used as light-emitting-layer host materials, and used for transport layers and injection layers.

Group B is a group of compounds that satisfy (A), (B), which are described above, and (C1) to (E1) described below. The bonds between the basic skeletons and the substituents are carbon-carbon bonds; thus, the compounds of group B also have excellent durability characteristics.

(C1) A group that does not form a ring out of R₁ to R₄ is a hydrogen atom, an aryl group, or a heterocyclic group bonded through a carbon atom.

(D1) R₅, R₇, R₈, and R₁₀ are each a hydrogen atom or a heterocyclic group bonded through a carbon atom.

(E1) A substituent on an aromatic ring formed by taking at least one of the sets of R₁ and R₂, R₂ and R₃, and R₃ and R₄ together is an aryl group or a heterocyclic group bonded through a carbon atom.

C1 to C10 belonging to group C are compounds that satisfy (A), (B), which are described above, and (C2) to (E2) described below. The bonds between the basic skeletons and the substituents in the compounds include hetero-bonds, which are weaker than carbon-carbon bonds; thus, the compounds have slightly shorter operating lives than groups A and B.

(C2) A group that does not form a ring out of R₁ to R₄ is a hydrogen atom, an aryl group, or a group bonded through an atom other than a carbon atom.

(D2) R₅, R₇, R₈, and R₁₀ are each a hydrogen atom or a group bonded through an atom other than a carbon atom.

(E2) A substituent on an aromatic ring formed by taking at least one of the sets of R₁ and R₂, R₂ and R₃, and R₃ and R₄ together is a group bonded through an atom other than a carbon atom.

Organic Light-Emitting Device

An organic light-emitting device according to an embodiment of the present disclosure will be described below.

The organic light-emitting device according to the embodiment at least includes a first electrode, a second electrode, which are a pair of electrodes, and an organic compound layer disposed between these electrodes. One of the first electrode and the second electrode may be an anode, and the other may be a cathode. In the organic light-emitting device according to the embodiment, the organic compound layer may be formed of a single layer or a multilayer stack including multiple layers as long as it includes a light-emitting layer.

In the case where the organic compound layer is formed of a multilayer stack including multiple layers, the organic compound layer may include, in addition to the light-emitting layer, a hole injection layer, a hole transport layer, an electron-blocking layer, a hole/exciton-blocking layer, an electron transport layer, an electron injection layer, and so forth. The light-emitting layer may be formed of a single layer or a multilayer stack including multiple layers.

Specific examples of the configuration of the organic light-emitting device include configurations (1) to (6) described below:

(1) (substrate/)anode/light-emitting layer/electron injection layer/cathode;
(2) (substrate/)anode/hole transport layer/electron transport layer/electron injection layer/cathode;
(3) (substrate/)anode/hole transport layer/light-emitting layer/electron transport layer/electron injection layer/cathode;
(4) (substrate/)anode/hole injection layer/hole transport layer/light-emitting layer/electron transport layer/electron injection layer/cathode;
(5) (substrate/)anode/hole transport layer/light-emitting layer/blocking layer/electron transport layer/electron injection layer/cathode; and
(6) (substrate/)anode/hole injection layer/hole transport layer/light-emitting layer/blocking layer/electron transport layer/electron injection layer/cathode.

In the organic light-emitting device according to the embodiment, the organic compound layer includes at least one layer containing the organic compound according to the embodiment. Specifically, the organic compound according to the embodiment is contained in any of the above-described hole injection layer, hole transport layer, electron-blocking layer, light-emitting layer, hole/exciton-blocking layer, electron transport layer, electron injection layer, and so forth. The organic compound according to the embodiment can be contained in the light-emitting layer.

In the organic light-emitting device according to the embodiment, in the case where the organic compound according to the embodiment is contained in the light-emitting layer, the light-emitting layer may consist of only the organic compound according to the embodiment or may be composed of the organic compound according to the embodiment and another compound. In the case where the light-emitting layer is composed of the organic compound according to the embodiment and another compound, the organic compound according to the embodiment may be used as a host in the light-emitting layer or a guest therein. The organic compound can be used as a guest. The organic compound may be used as an assist material that can be contained in the light-emitting layer. The term “host” used here refers to a compound having the highest proportion by mass in compounds constituting the light-emitting layer. The term “guest” refers to a compound that has a lower proportion by mass than the host in the compounds constituting the light-emitting layer and that is responsible for main light emission. The term “assist material” refers to a compound that has a lower proportion by mass than the host in the compounds constituting the light-emitting layer and that assists the light emission of the guest. The assist material is also referred to as a “second host”.

In the case where the organic compound according to the embodiment is used as a guest in the light-emitting layer, the concentration of the guest is preferably 0.01% or more by mass and 20% or less by mass, more preferably 0.1% or more by mass and 5% or less by mass with respect to the entire light-emitting layer.

Additionally, in the case where the organic compound according to the embodiment is used as a guest in the light-emitting layer, a material having a higher LUMO level than the organic compound according to the embodiment (a material having a LUMO level closer to the vacuum level) can be used as a host. The reason for this is that in the case where the material having a higher LUMO level than the organic compound according to the embodiment is used as a host, the organic compound according to the embodiment can receive more electrons supplied to the host in the light-emitting layer. The use of the organic compound according to the embodiment as a guest can further improve the chromaticity during light emission. For example, shortening the emission spectrum wavelength of the basic skeleton can bring the chromaticity of blue-light emission close to the blue chromaticity specified in sRGB to expand the color gamut.

The organic compound according to the embodiment is used as a host or guest in the light-emitting layer, in particular, as a guest in the light-emitting layer. The light-emitting layer may be formed of a single layer or multiple layers and may contain a light-emitting material that emits another emission color. The term “multiple layers” refers to a state in which a light-emitting layer and another light-emitting layer are stacked. In this case, the emission color of the organic light-emitting device is not particularly limited. More specifically, the emission color of the organic light-emitting device is not limited to blue, and may be white or a neutral color. In the case of white, another light-emitting layer emits light other than blue light, i.e., red or green light. Additionally, each light-emitting layer may emit blue, green, or red light. Regarding a method for forming a film, at least the light-emitting layer can be formed by a vacuum evaporation method.

The organic compound according to the embodiment can be used as a material for an organic compound layer other than the light-emitting layer in the organic light-emitting device according to the embodiment. Specifically, the organic compound according to the embodiment may be used as a material for the electron transport layer, the electron injection layer, the hole transport layer, the hole injection layer, the hole-blocking layer, and so forth.

For example, a hole injection compound, a hole transport compound, a compound to be used as a host, a light-emitting compound, an electron injection compound, or an electron transport compound, which is known and has a low or high molecular weight, can be used together with the organic compound according to the embodiment, as needed. Examples of these compounds will be described below.

As a hole injection-transport material, a material having a high hole mobility can be used so as to facilitate the injection of holes from the anode and to transport the injected holes to the light-emitting layer. To reduce a deterioration in film quality, such as crystallization, in the organic light-emitting device, a material having a high glass transition temperature can be used. Examples of a low- or high-molecular-weight material having the ability to inject and transport holes include triarylamine derivatives, aryl carbazole derivatives, phenylenediamine derivatives, stilbene derivatives, phthalocyanine derivatives, porphyrin derivatives, poly(vinyl carbazole), polythiophene, and other conductive polymers. Furthermore, the hole injection-transport material can be used for the electron-blocking layer. Non-limiting specific examples of a compound used as the hole injection-transport material will be illustrated below.

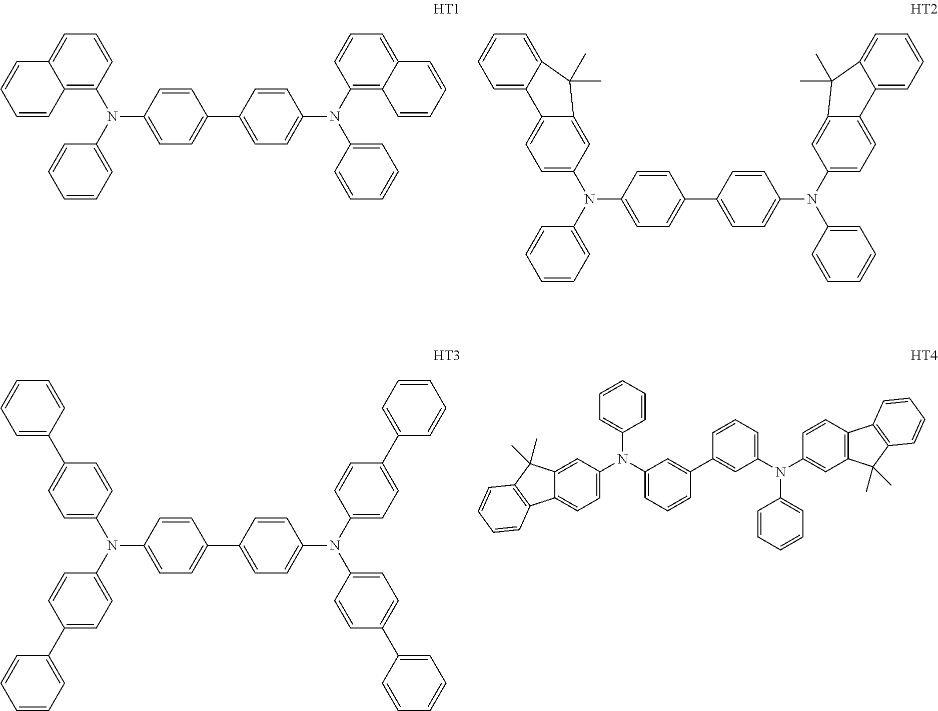

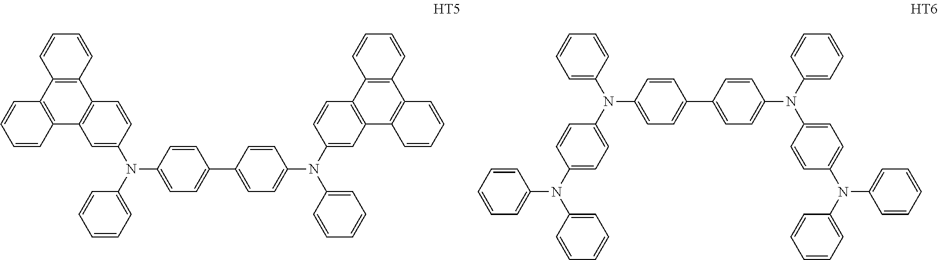 [SMILES: c1ccc(N(c2ccc(-c3ccc(N(c4ccccc4)c4ccc5c6ccccc6c6ccccc6c5c4)cc3)cc2)c2ccc3c4ccccc4c4ccccc4c3c2)cc1 ; c1ccc(N(c2ccccc2)c2ccc(N(c3ccccc3)c3ccc(-c4ccc(N(c5ccccc5)c5ccc(N(c6ccccc6)c6ccccc6)cc5)cc4)cc3)cc2)cc1]

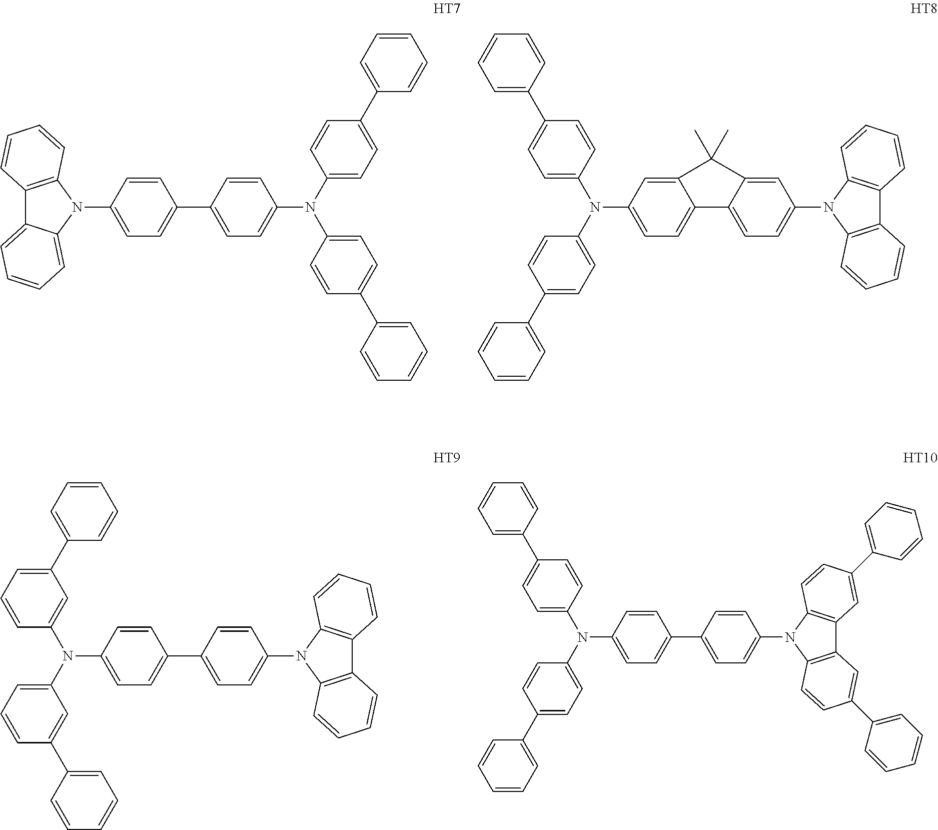 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccc(-c4ccccc4)cc3)c3ccc(-c4ccccc4)cc3)ccc2-c2ccc(-n3c4ccccc4c4ccccc43)cc21 ; c1ccc(-c2cccc(N(c3ccc(-c4ccc(-n5c6ccccc6c6ccccc65)cc4)cc3)c3cccc(-c4ccccc4)c3)c2)cc1 ; c1ccc(-c2ccc(N(c3ccc(-c4ccccc4)cc3)c3ccc(-c4ccc(-n5c6ccc(-c7ccccc7)cc6c6cc(-c7ccccc7)ccc65)cc4)cc3)cc2)cc1]

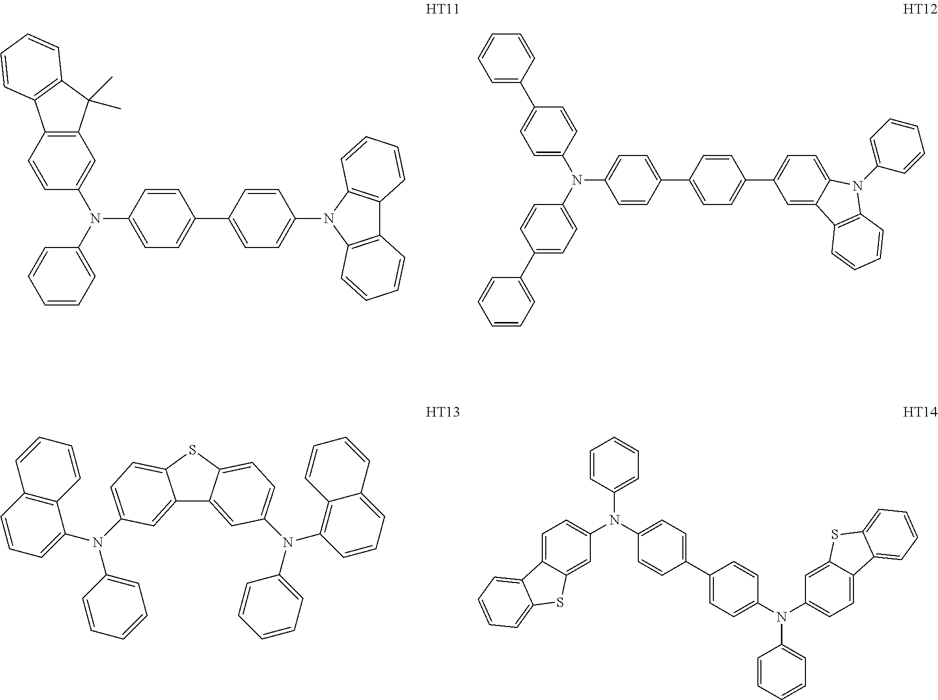 [SMILES: CC1(C)c2ccccc2-c2ccc(N(c3ccccc3)c3ccc(-c4ccc(-n5c6ccccc6c6ccccc65)cc4)cc3)cc21 ; c1ccc(-c2ccc(N(c3ccc(-c4ccccc4)cc3)c3ccc(-c4ccc(-c5ccc6c(c5)c5ccccc5n6-c5ccccc5)cc4)cc3)cc2)cc1 ; c1ccc(N(c2ccc3sc4ccc(N(c5ccccc5)c5cccc6ccccc56)cc4c3c2)c2cccc3ccccc23)cc1 ; c1ccc(N(c2ccc(-c3ccc(N(c4ccccc4)c4ccc5c(c4)sc4ccccc45)cc3)cc2)c2ccc3c(c2)sc2ccccc23)cc1]

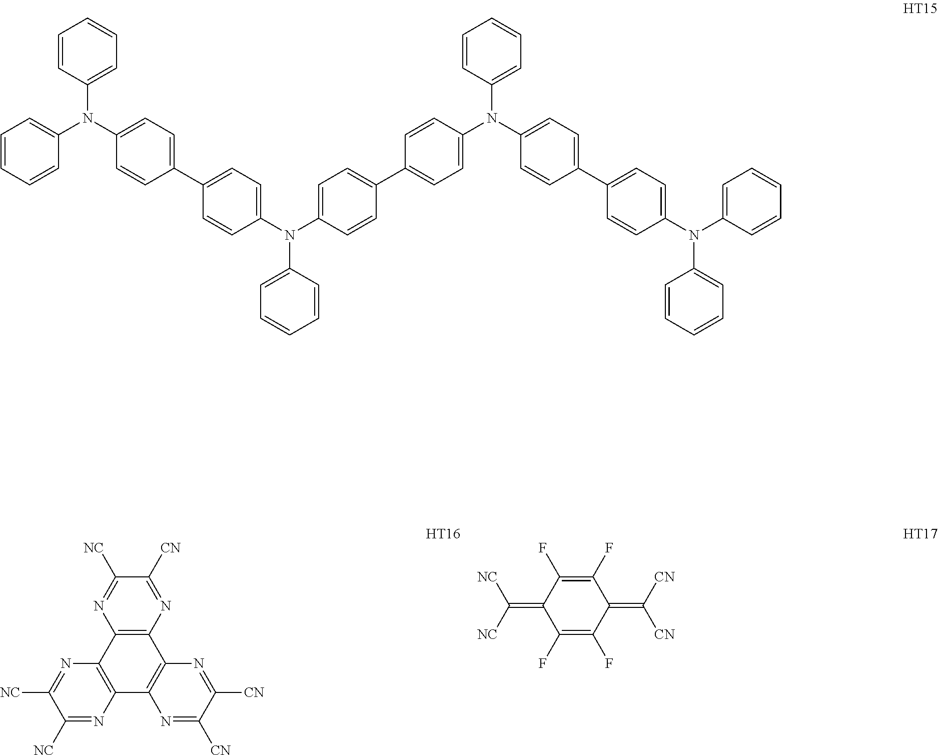 [SMILES: c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1 ; N#Cc1nc2c3nc(C#N)c(C#N)nc3c3nc(C#N)c(C#N)nc3c2nc1C#N ; N#CC(C#N)=c1c(F)c(F)c(=C(C#N)C#N)c(F)c1F]

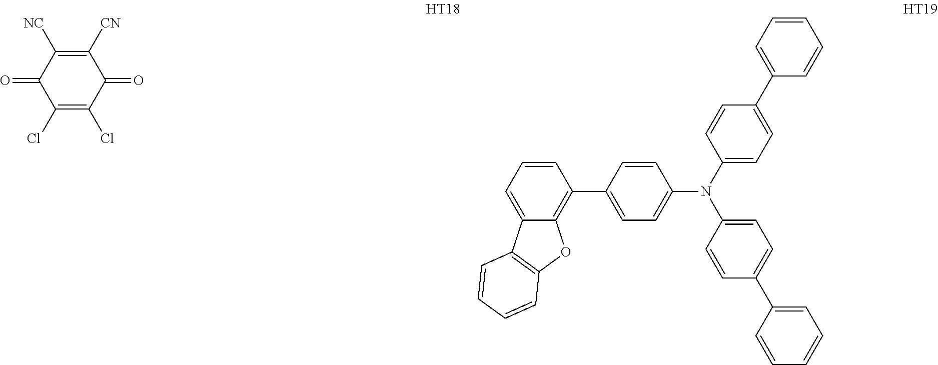 [SMILES: N#CC1=C(C#N)C(=O)C(Cl)=C(Cl)C1=O ; c1ccc(-c2ccc(N(c3ccc(-c4ccccc4)cc3)c3ccc(-c4cccc5c4oc4ccccc45)cc3)cc2)cc1]

Examples of a light-emitting material mainly associated with a light-emitting function include, in addition to the organic compound represented by formula [1], condensed-ring compounds, such as fluorene derivatives, naphthalene derivatives, pyrene derivatives, perylene derivatives, tetracene derivatives, anthracene compounds, and rubrene, quinacridone derivatives, coumarin derivatives, stilbene derivatives, organoaluminum complexes, such as tris(8-quinolinolato)aluminum, iridium complexes, platinum complexes, rhenium complexes, copper complexes, europium complexes, ruthenium complexes, and polymer derivatives, such as poly(phenylene vinylene)derivatives, polyfluorene derivatives, and polyphenylene derivatives.

In the case where a mixed layer containing the organic compound according to the embodiment and another light-emitting material is formed or where light-emitting layers are stacked, the another light-emitting material can have low HOMO/LUMO energy levels. The reason for this is that in the case of high HOMO/LUMO energy levels, the material may form an exciplex with the organic compound according to the embodiment to form a quenching component or trap level.

Non-limiting specific examples of a compound used for a light-emitting material will be illustrated below.

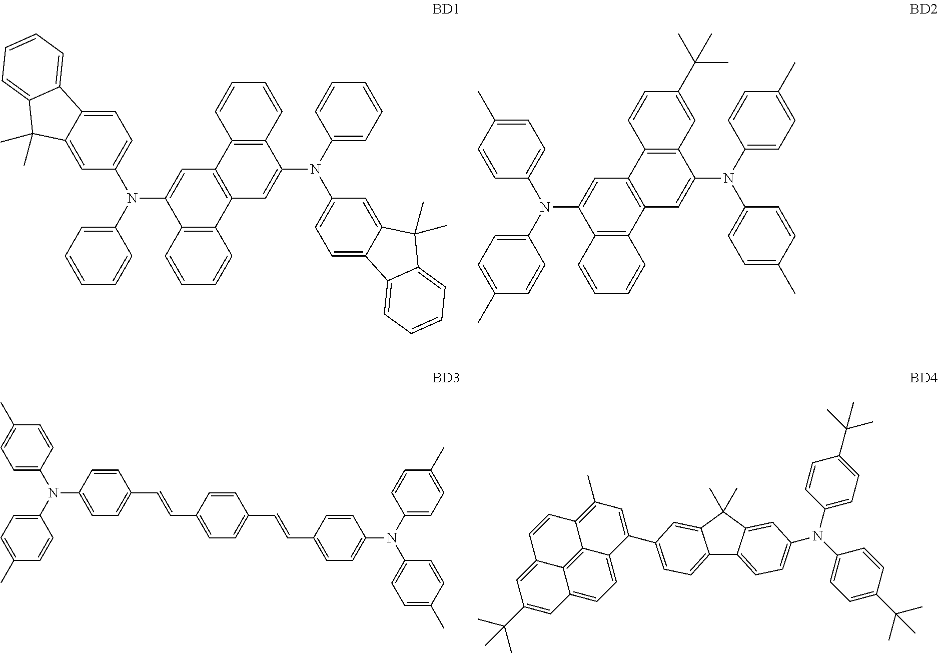

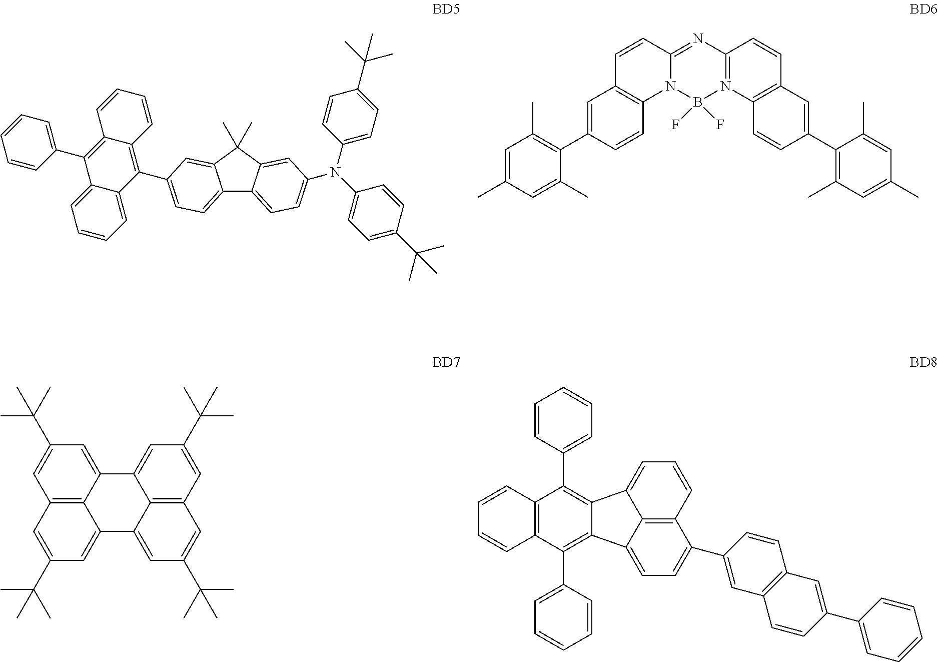

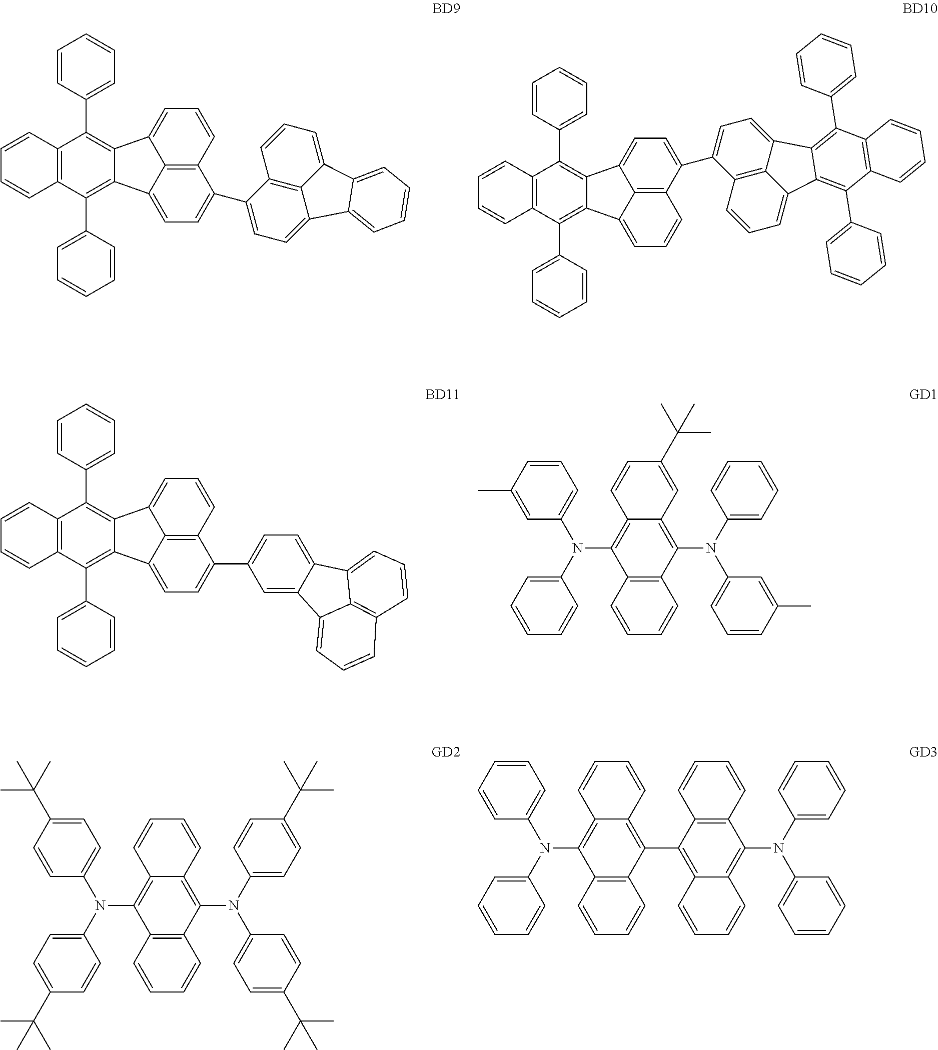

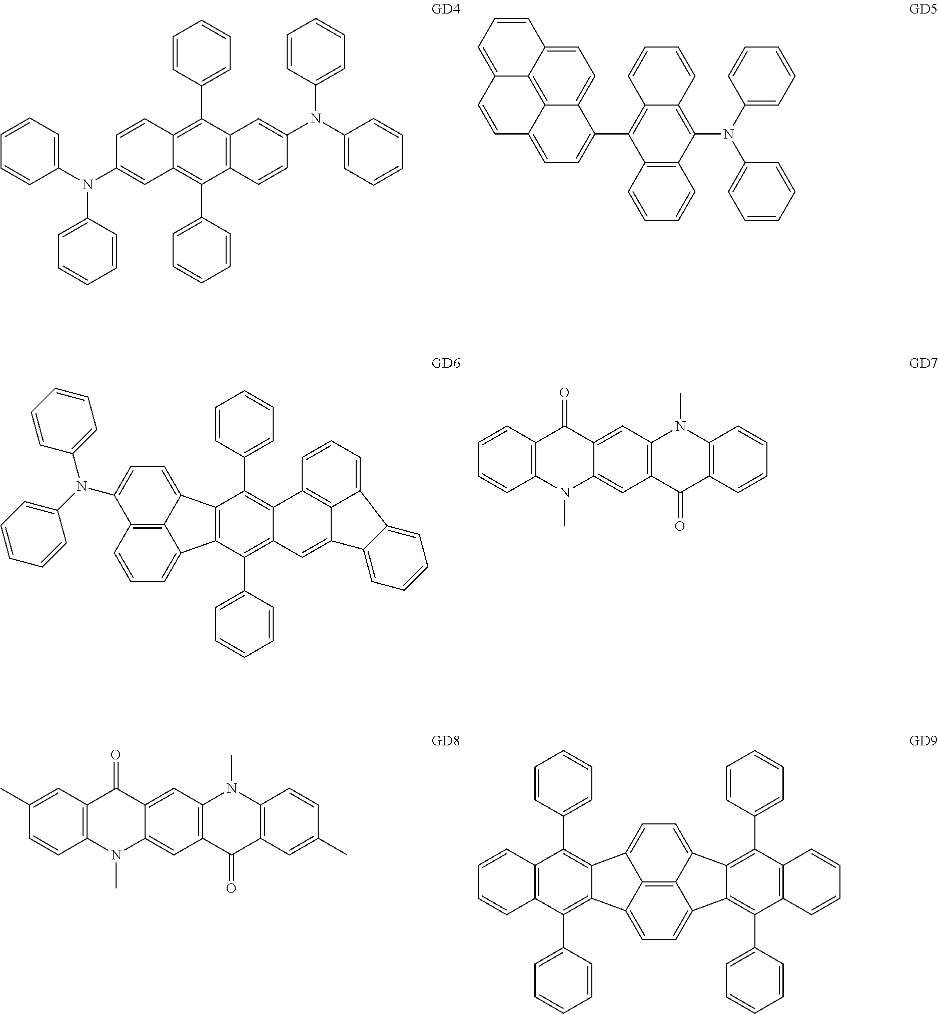

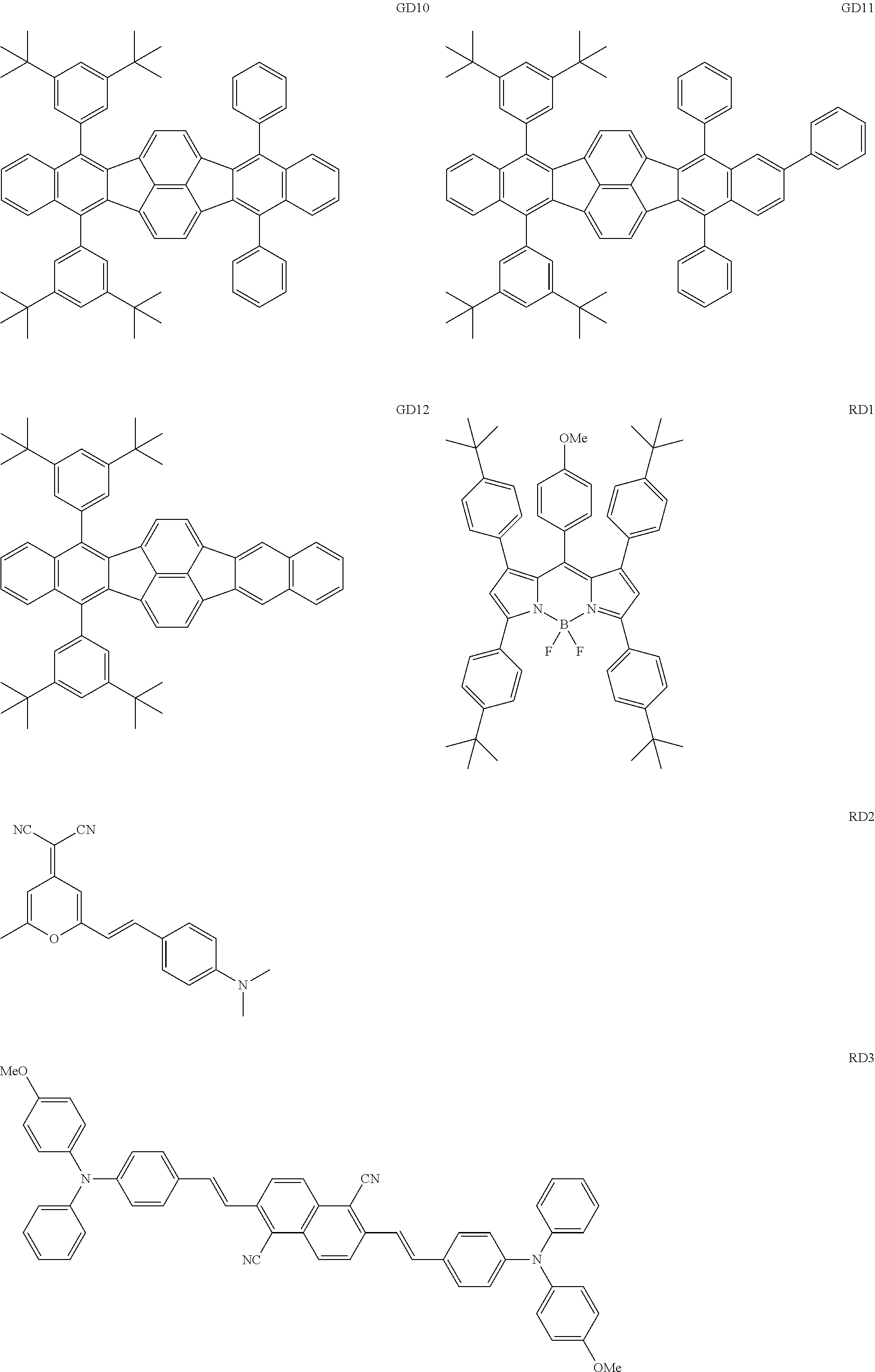

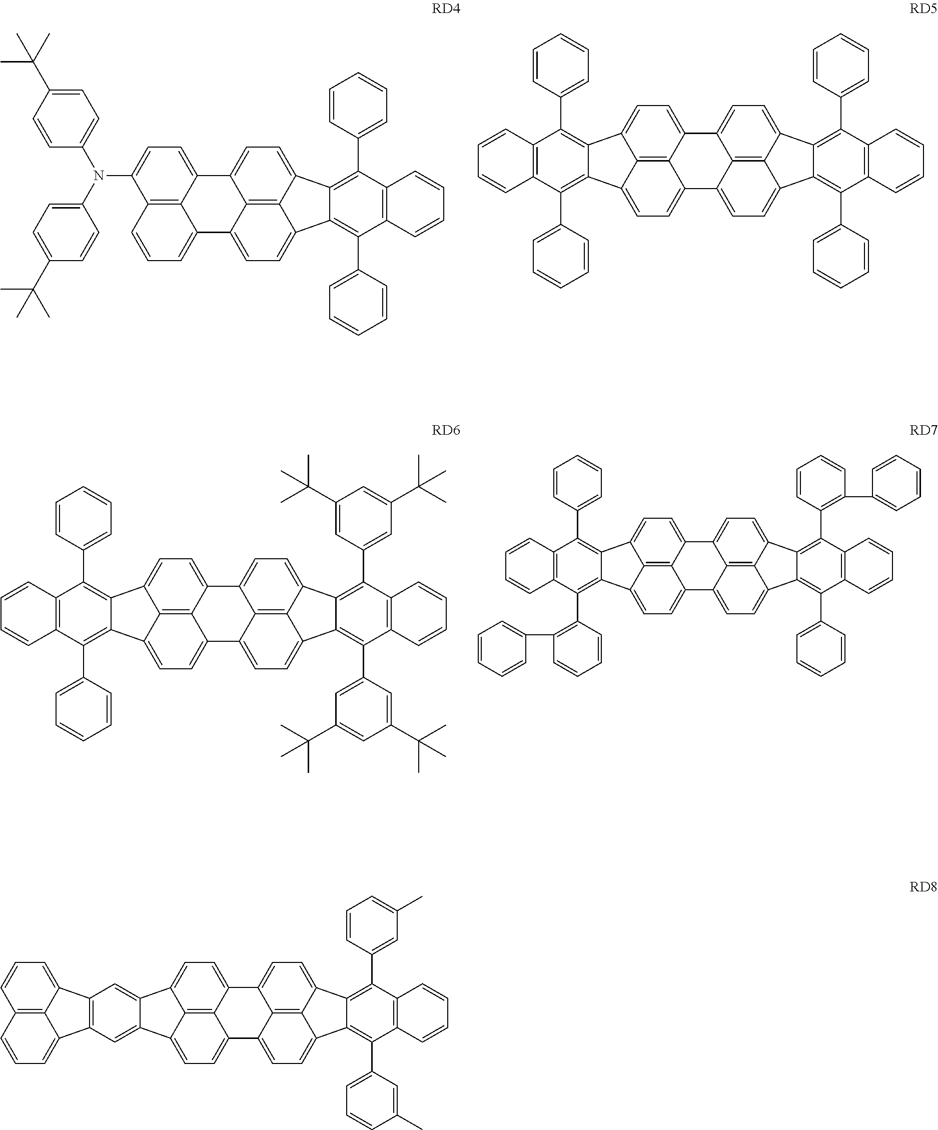

Examples of a host or an assist material in the light-emitting layer include aromatic hydrocarbon compounds and derivatives thereof, carbazole derivatives, dibenzofuran derivatives, dibenzothiophene derivatives, organoaluminum complexes, such as tris(8-quinolinolato)aluminum, and organoberyllium complexes. In particular, the host material can have an anthracene skeleton, a tetracene skeleton, a perylene skeleton, or a pyrene skeleton in its molecular skeleton. This is because the material consists of carbon and hydrogen as described above and has an Si energy that can cause sufficient energy transfer to the organic compound according to the embodiment. Non-limiting specific examples of a compound used as a host or an assist material in the light-emitting layer will be illustrated below.

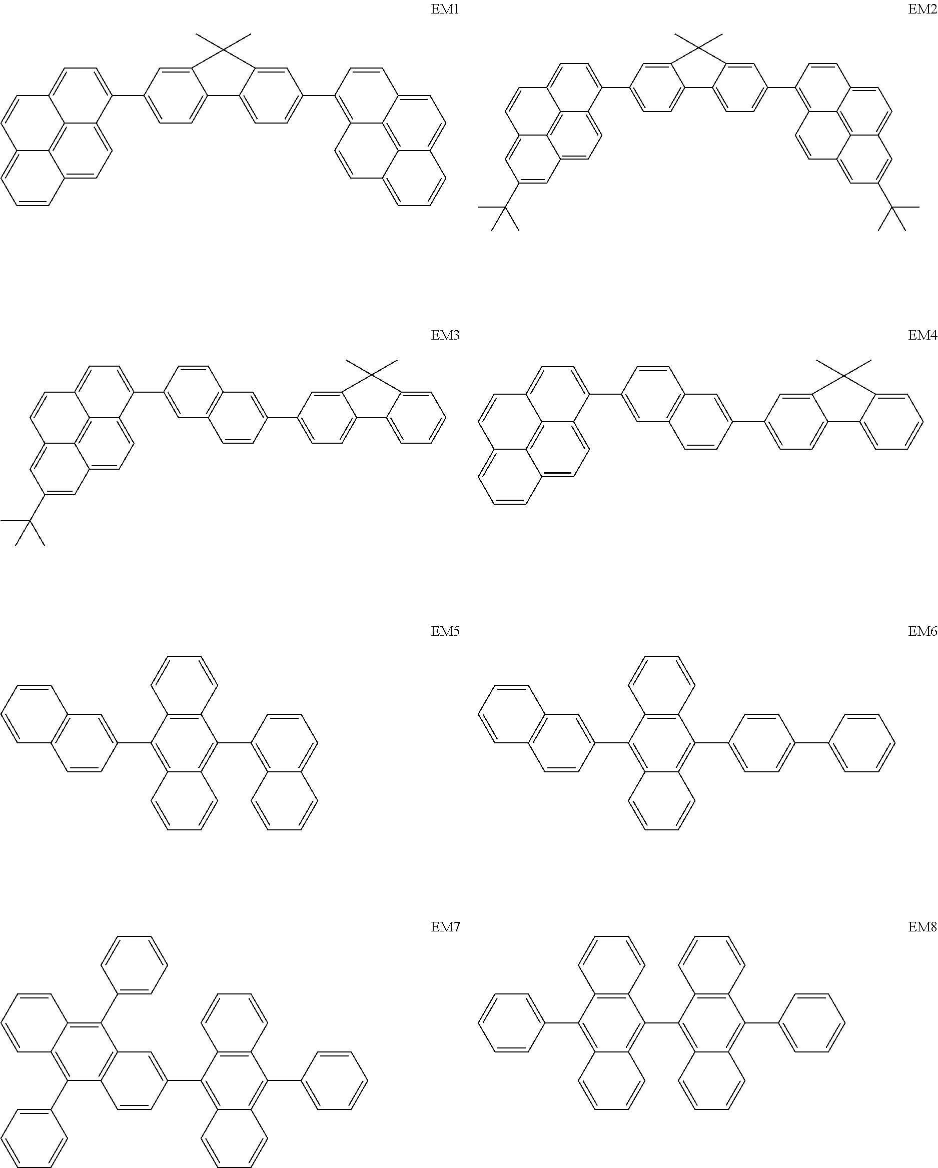

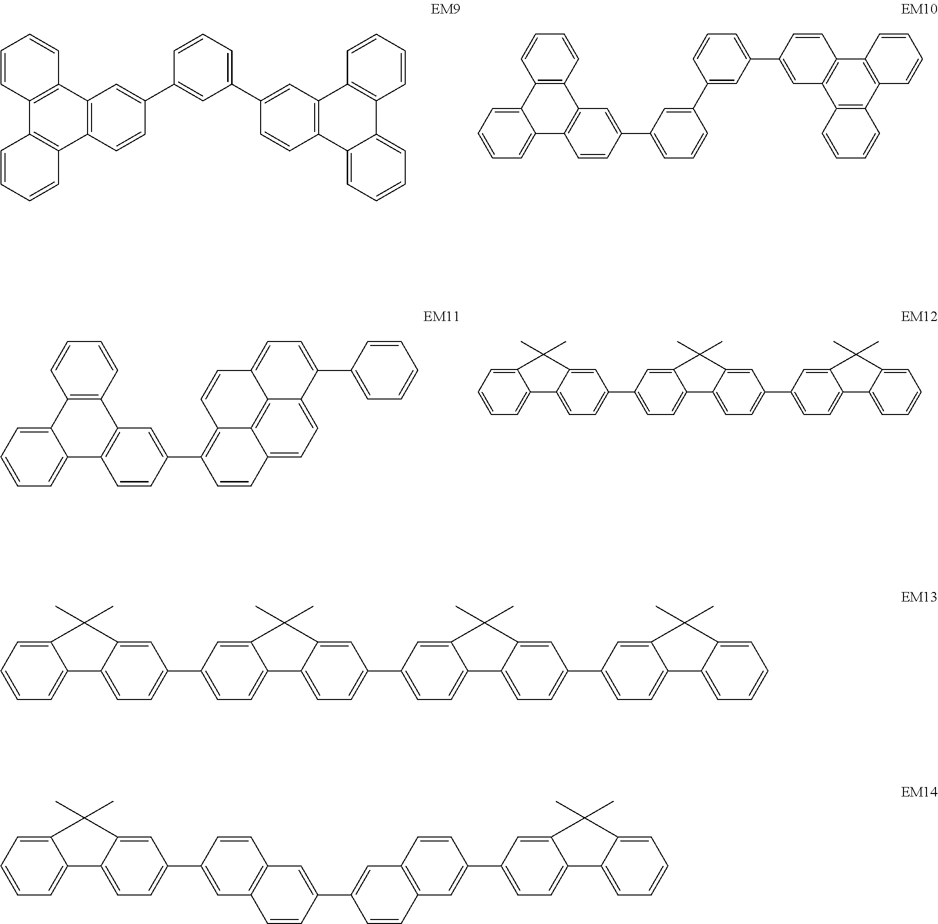

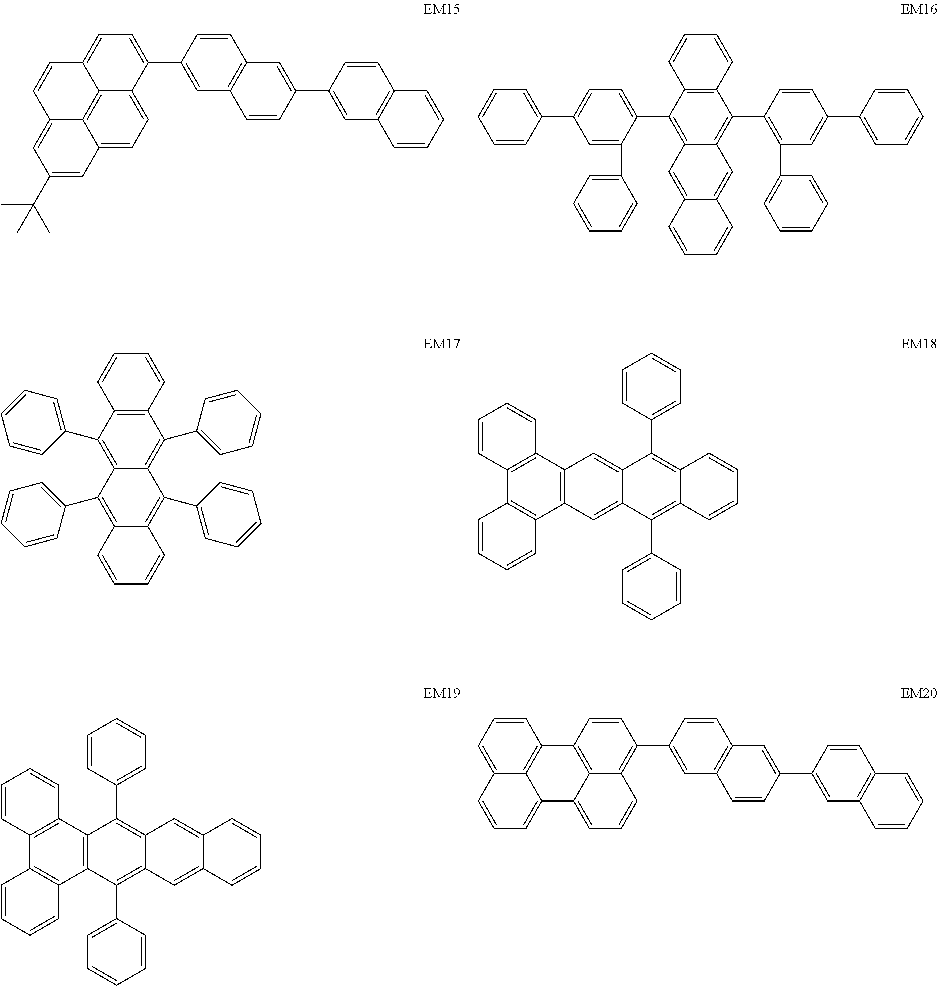

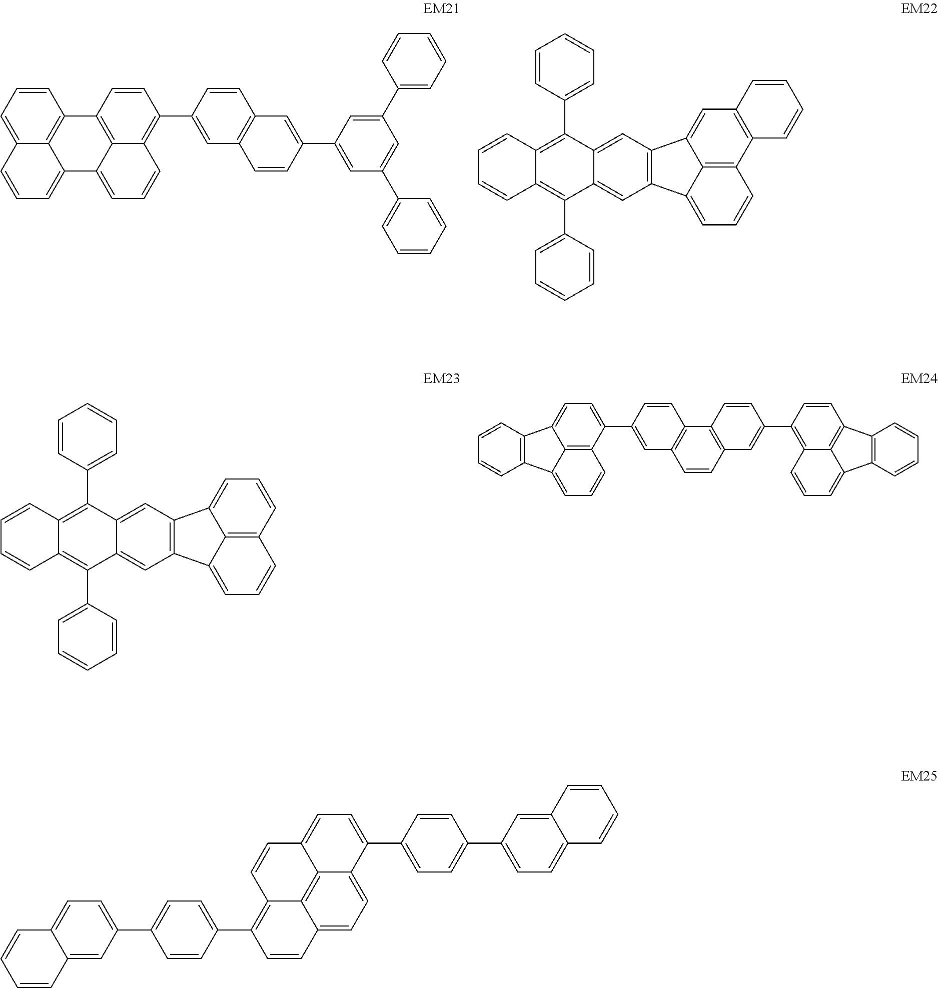

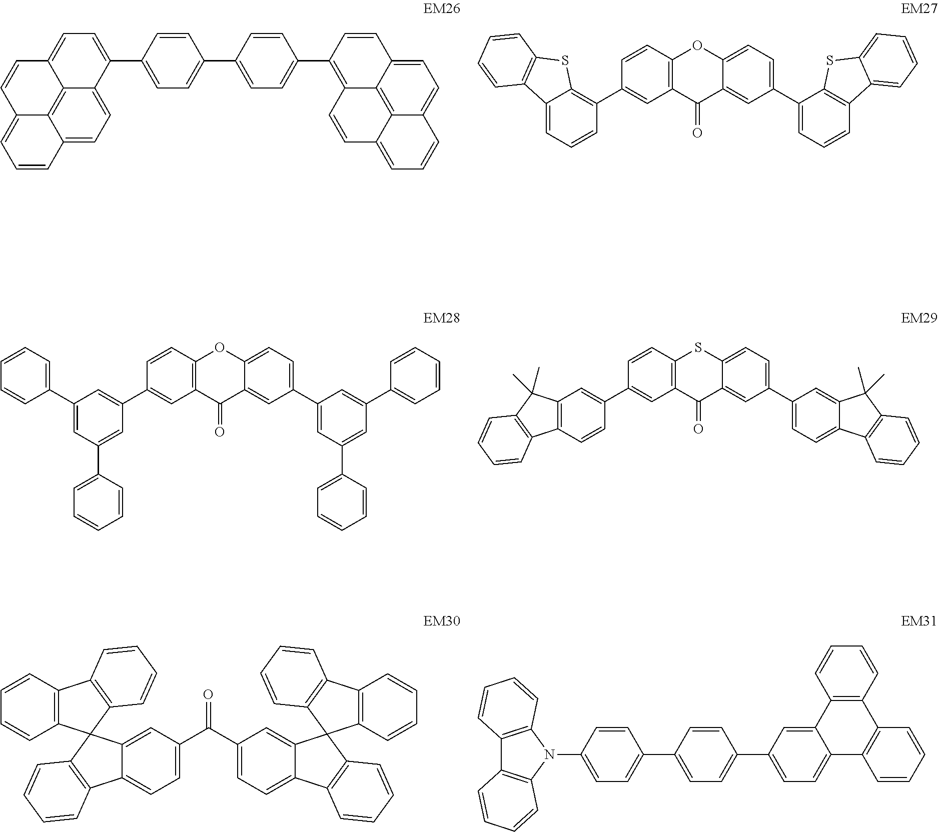

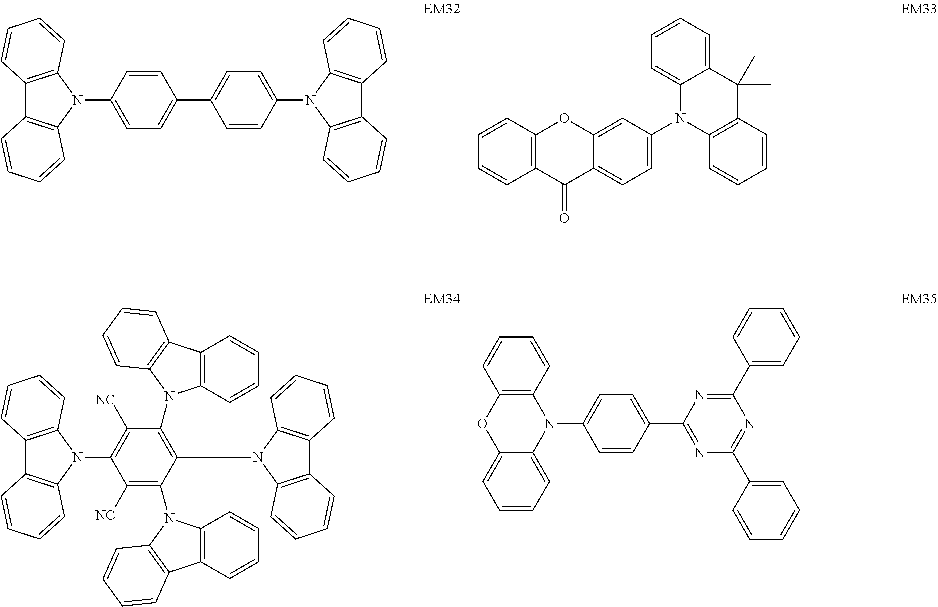 [SMILES: c1ccc2c(c1)c1ccccc1n2-c1ccc(-c2ccc(-n3c4ccccc4c4ccccc43)cc2)cc1 ; CC1(C)c2ccccc2N(c2ccc3c(=O)c4ccccc4oc3c2)c2ccccc21 ; N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21 ; c1ccc(-c2nc(-c3ccccc3)nc(-c3ccc(N4c5ccccc5Oc5ccccc54)cc3)n2)cc1]

The electron transport material can be freely-selected from materials capable of transporting electrons injected from the cathode to the light-emitting layer and is selected in consideration of, for example, the balance with the hole mobility of the hole transport material. Examples of a material having the ability to transport electrons include oxadiazole derivatives, oxazole derivatives, pyrazine derivatives, triazole derivatives, triazine derivatives, quinoline derivatives, quinoxaline derivatives, phenanthroline derivatives, organoaluminum complexes, and condensed-ring compounds, such as fluorene derivatives, naphthalene derivatives, chrysene derivatives, and anthracene derivatives. The electron transport materials can be used for the hole-blocking layer. Non-limiting specific examples of a compound used as the electron transport material will be illustrated below.

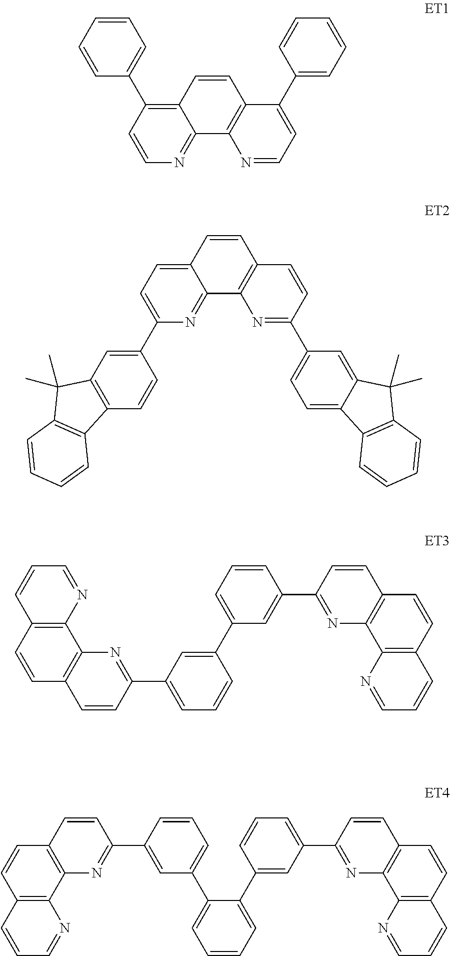

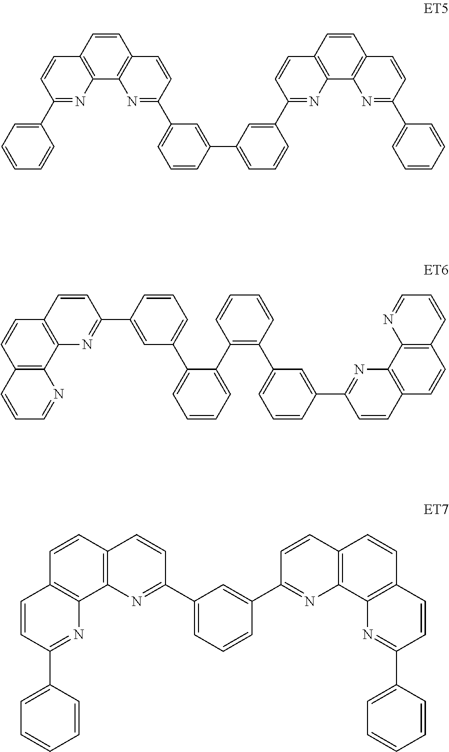 [SMILES: c1ccc(-c2ccc3ccc4ccc(-c5cccc(-c6cccc(-c7ccc8ccc9ccc(-c%10ccccc%10)nc9c8n7)c6)c5)nc4c3n2)cc1 ; c1cc(-c2ccc3ccc4cccnc4c3n2)cc(-c2ccccc2-c2ccccc2-c2cccc(-c3ccc4ccc5cccnc5c4n3)c2)c1 ; c1ccc(-c2ccc3ccc4ccc(-c5cccc(-c6ccc7ccc8ccc(-c9ccccc9)nc8c7n6)c5)nc4c3n2)cc1]

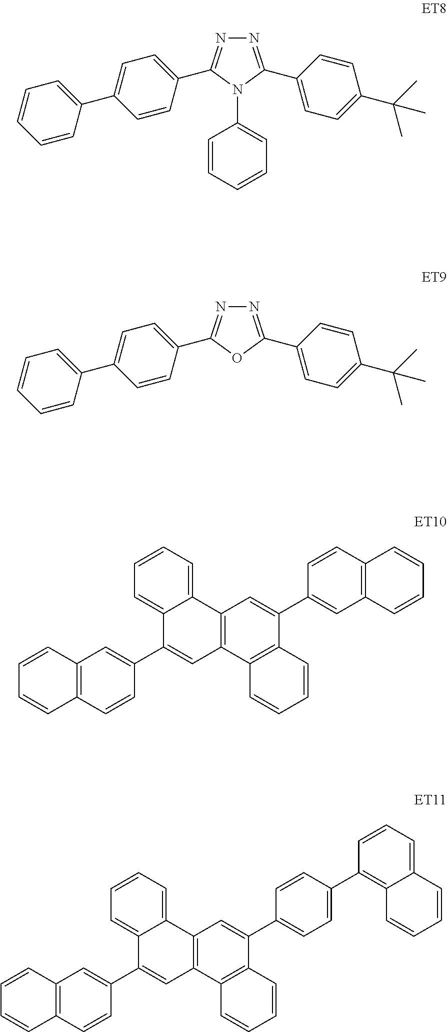

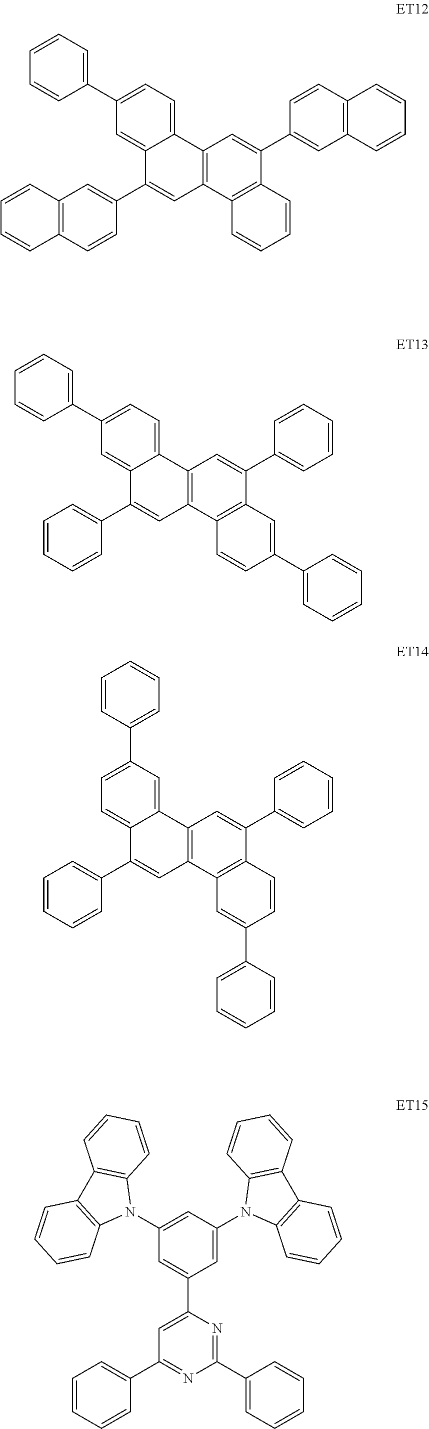

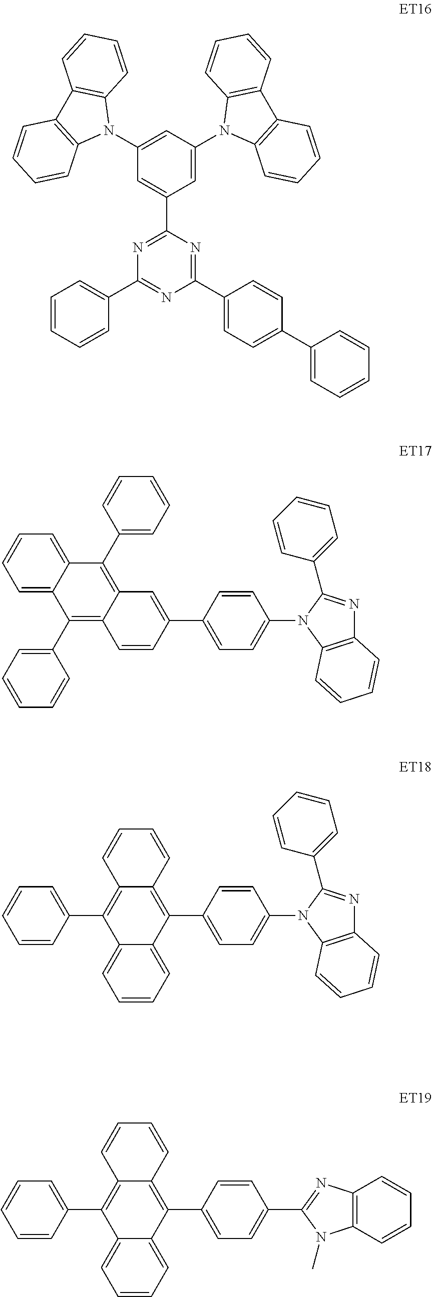 [SMILES: c1ccc(-c2ccc(-c3nc(-c4ccccc4)nc(-c4cc(-n5c6ccccc6c6ccccc65)cc(-n5c6ccccc6c6ccccc65)c4)n3)cc2)cc1 ; c1ccc(-c2c3ccccc3c(-c3ccccc3)c3cc(-c4ccc(-n5c(-c6ccccc6)nc6ccccc65)cc4)ccc23)cc1 ; c1ccc(-c2c3ccccc3c(-c3ccc(-n4c(-c5ccccc5)nc5ccccc54)cc3)c3ccccc23)cc1 ; Cn1c(-c2ccc(-c3c4ccccc4c(-c4ccccc4)c4ccccc34)cc2)nc2ccccc21]

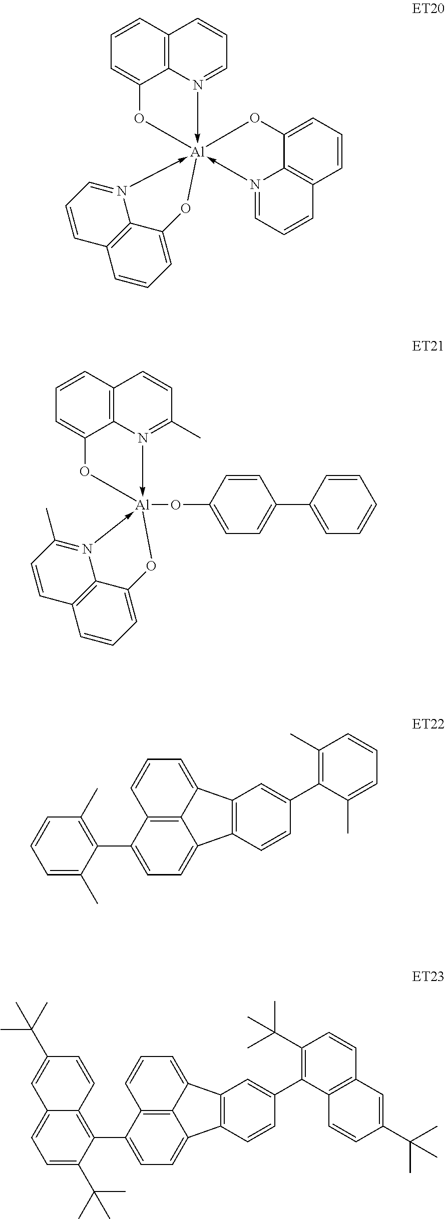

Configuration of Organic Light-Emitting Device

The organic light-emitting device is provided by disposing an anode, the organic compound layer, and a cathode on a substrate. A protective layer, a color filter, and so forth may be disposed on the cathode. In the case of disposing the color filter, a planarization layer may be disposed between the protective layer and the color filter. The planarization layer can be composed of, for example, an acrylic resin.

Substrate

Examples of the substrate include silicon wafers, quartz substrates, glass substrates, resin substrates, and metal substrates. The substrate may include switching devices such as a transistor, a line, and an insulating layer thereon. As the insulating layer, any material can be used as long as a contact hole can be formed to establish the electrical connection between the anode and the line and as long as insulation with a non-connected line can be ensured. For example, a resin such as polyimide, silicon oxide, or silicon nitride can be used.

Electrode

Regarding an electrode, a pair of electrodes can be used. The pair of electrodes may be an anode and a cathode. In the case where an electric field is applied in the direction in which the organic light emitting element emits light, an electrode having a higher potential is the anode, and the other is the cathode. It can also be said that the electrode that supplies holes to the light-emitting layer is the anode and that the electrode that supplies electrons is the cathode.

As the constituent material of the anode, a material having a work function as high as possible can be used. Examples of the material that can be used include elemental metals, such as gold, platinum, silver, copper, nickel, palladium, cobalt, selenium, vanadium, and tungsten, mixtures thereof, alloys of combinations thereof, and metal oxides, such as tin oxide, zinc oxide, indium oxide, indium-tin oxide (ITO), and indium-zinc oxide. Additionally, conductive polymers, such as polyaniline, polypyrrole, and polythiophene, may be used.

These electrode materials may be used alone or in combination of two or more. The anode may be formed of a single layer or multiple layers.

In the case where the anode is used as a reflective electrode, for example, chromium, aluminum, silver, titanium, tungsten, molybdenum, an alloy thereof, or a stack thereof may be used. In the case where the anode is used as a transparent electrode, a transparent conductive oxide layer composed of, for example, indium-tin oxide (ITO) or indium-zinc oxide may be used; however, the anode is not limited thereto. The electrode may be formed by photolithography.

As the constituent material of the cathode, a material having a lower work function can be used. Examples thereof include elemental metals, such as alkali metals, e.g., lithium, alkaline-earth metals, e.g., calcium, aluminum, titanium, manganese, silver, lead, and chromium, and mixtures containing them. Alloys of combinations thereof may also be used. For example, magnesium-silver, aluminum-lithium, aluminum-magnesium, silver-copper, and zinc-silver may be used. Metal oxides, such as indium-tin oxide (ITO), may also be used. These electrode materials may be used alone or in combination of two or more. The cathode may have a single-layer structure or a multilayer structure. In particular, silver can be used. To reduce the aggregation of silver, a silver alloy can be used. Any alloy ratio may be used as long as the aggregation of silver can be reduced. For example, 1:1 may be used.

A top emission device may be provided using the cathode formed of a conductive oxide layer composed of, for example, ITO. A bottom emission device may be provided using the cathode formed of a reflective electrode composed of, for example, aluminum (Al). The cathode is not particularly limited. Any method for forming the cathode may be used. For example, a direct-current or alternating-current sputtering technique can be employed because good film coverage is obtained and thus the resistance is easily reduced.

Protective Layer

A protective layer may be disposed on the cathode. For example, a glass member provided with a moisture absorbent can be bonded to the cathode to reduce the entry of, for example, water into the organic compound layer, thereby suppressing the occurrence of display defects. In another embodiment, a passivation film composed of, for example, silicon nitride may be disposed on the cathode to reduce the entry of, for example, water into the organic compound layer. For example, after the formation of the cathode, the substrate may be transported to another chamber without breaking the vacuum, and a silicon nitride film having a thickness of 2 μm may be formed by a chemical vapor deposition (CVD) method to provide a protective layer. After the film deposition by the CVD method, a protective layer may be formed by an atomic layer deposition (ALD) method.

Color Filter

A color filter may be disposed on the protective layer. For example, a color filter may be disposed on another substrate in consideration of the size of the organic light-emitting device and bonded to the substrate provided with the organic light-emitting device. A color filter may be formed by patterning on the protective film using photolithography. The color filter may be composed of a polymer.

Planarization Layer

A planarization layer may be disposed between the color filter and the protective layer. The planarization layer may be composed of an organic compound. A low- or high-molecular-weight organic compound may be used. A high-molecular-weight organic compound can be used.

The planarization layers may be disposed above and below (or on) the color filter and may be composed of the same or different materials. Specific examples thereof include poly(vinyl carbazole) resins, polycarbonate resins, polyester resins, acrylonitrile butadiene styrene (ABS) resins, acrylic resins, polyimide resins, phenolic resins, epoxy resins, silicone resins, and urea resins.

Opposite Substrate

An opposite substrate may be disposed on the planarization layer. The opposite substrate is disposed at a position corresponding to the substrate described above and thus is called an opposite substrate. The opposite substrate may be composed of the same material as the substrate described above.

Organic Layer

The organic compound layer, such as the hole injection layer, the hole transport layer, the electron-blocking layer, the light-emitting layer, the hole-blocking layer, the electron transport layer, or the electron injection layer, included in the organic light-emitting device according to an embodiment of the present disclosure is formed by a method described below.

For the organic compound layer included in the organic light-emitting device according to an embodiment of the present disclosure, a dry process, such as a vacuum evaporation method, an ionized evaporation method, sputtering, or plasma, may be employed. Alternatively, instead of the dry process, it is also possible to employ a wet process in which a material is dissolved in an appropriate solvent and then a film is formed by a known coating method, such as spin coating, dipping, a casting method, a Langmuir-Blodgett (LB) technique, or an ink jet method.

In the case where the layer is formed by, for example, the vacuum evaporation method or the solution coating method, crystallization and so forth are less likely to occur, and good stability with time is obtained. In the case of forming a film by the coating method, the film may be formed in combination with an appropriate binder resin.

Non-limiting examples of the binder resin include poly(vinyl carbazole) resins, polycarbonate resins, polyester resins, acrylonitrile butadiene styrene (ABS) resins, acrylic resins, polyimide resins, phenolic resins, epoxy resins, silicone resins, and urea resins.

These binder resins may be used alone as a homopolymer or copolymer or in combination as a mixture. Furthermore, additives, such as a known plasticizer, antioxidant, and ultraviolet absorber, may be used, as needed.

Application of Organic Light-Emitting Device According to an Embodiment of the Present Disclosure

The organic light-emitting device according to an embodiment of the present disclosure can be used as component member of a display device or a lighting device. Other applications include exposure light sources for electrophotographic image forming apparatuses, backlights for liquid crystal displays, and light-emitting devices including white light sources and color filters.

The display device may be an image information-processing device having an image input unit that receives image information from an area or linear CCD sensor, a memory card, or any other source, an information-processing unit that processes the input information, and a display unit that displays the input image. The display device includes multiple pixels, and at least one of the multiple pixels may include the organic light-emitting device according to the embodiment and a transistor coupled to the organic light-emitting device.

The display unit of an image pickup apparatus or an inkjet printer may have a touch-screen feature. The driving mode of the touch-screen feature may be, but is not limited to, an infrared mode, an electrostatic capacitive mode, a resistive film mode, or an electromagnetic inductive mode. The display device may also be used for a display unit of a multifunction printer.

The following describes a display device according to the embodiment with reference to the attached drawings. FIG. 2 is a schematic cross-sectional view illustrating an example of a display device including organic light-emitting devices and thin-film transistor (TFT) devices coupled to the respective organic light-emitting devices. Each of the TFT devices is an example of active devices.

A display device 10 illustrated in FIG. 2 includes a substrate 11 composed of, for example, glass and a moisture-proof film 12 disposed thereon, the moisture-proof film 12 being configured to protect TFT devices or the organic compound layers. Reference numeral 13 denotes a gate electrode composed of a metal. Reference numeral 14 denotes a gate insulating film. Reference numeral 15 denotes a semiconductor layer.

TFT devices 18 each include the semiconductor layer 15, a drain electrode 16, and a source electrode 17. An insulating film 19 is disposed on the TFT devices 18. An anode 21 included in an organic light-emitting device 26 is coupled to the source electrode 17 through a contact hole 20.

The way of electric coupling between the electrodes (the anode 21 and a cathode 23) included in each of the organic light-emitting devices 26 and the electrodes (the source electrode 17 and the drain electrode 16) included in a corresponding one of the TFT devices 18 is not limited to the configuration illustrated in FIG. 2. It is sufficient that one of the anode 21 and the cathode 23 is electrically coupled to one of the source electrode 17 and the drain electrode 16 of the TFT device 18.

In the display device 10 illustrated in FIG. 2, each organic compound layer 22 is illustrated as a single layer; however, the organic compound layer 22 may be formed of multiple layers. A first protective layer 24 and a second protective layer 25 are disposed on the cathodes 23 in order to reduce the deterioration of the organic light-emitting devices 26.

In the display device 10 illustrated in FIG. 2, the transistors are used as switching devices; however, metal-insulator-metal (MIM) devices may be used as switching devices.

The transistors used in the display device 10 illustrated in FIG. 2 are not limited to transistors formed using a single-crystal silicon wafer and may be thin-film transistors each having an active layer on the insulating surface of a substrate. Examples of the material of the active layer include single-crystal silicon, non-single-crystal silicon materials such as amorphous silicon and microcrystalline silicon, and non-single-crystal oxide semiconductors, such as indium-zinc oxide and indium-gallium-zinc oxide. Thin-film transistors are also referred to as TFT devices.

The transistors in the display device 10 illustrated in FIG. 2 may be formed in the substrate such as a Si substrate. The expression “formed in the substrate” indicates that the transistors are produced by processing the substrate such as a Si substrate. In the case where the transistors are formed in the substrate, the substrate and the transistors can be deemed to be integrally formed.

In the organic light-emitting device according to the embodiment, the luminance is controlled by the TFT devices, which are an example of switching devices; thus, an image can be displayed at respective luminance levels by arranging multiple organic light-emitting devices in the plane. The switching devices according to the embodiment are not limited to the TFT devices and may be low-temperature polysilicon transistors or active-matrix drivers formed on a substrate such as a Si substrate. The expression “on a substrate” can also be said to be “in the substrate”. Whether transistors are formed in the substrate or TFT devices are used is selected in accordance with the size of a display unit. For example, in the case where the display unit has a size of about 0.5 inches, organic light-emitting devices can be disposed on a Si substrate.

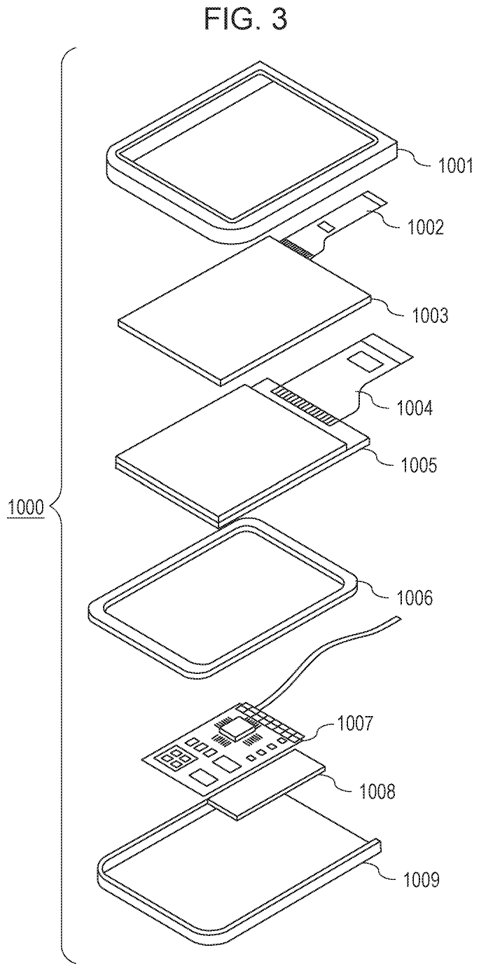

FIG. 3 is a schematic view illustrating an example of a display device according to the embodiment. A display device 1000 may include a touch screen 1003, a display panel 1005, a frame 1006, a circuit substrate 1007, and a battery 1008 disposed between an upper cover 1001 and a lower cover 1009. The touch screen 1003 and the display panel 1005 are coupled to flexible printed circuits FPCs 1002 and 1004, respectively. The circuit substrate 1007 includes printed transistors. The battery 1008 need not be provided unless the display device is a portable device. The battery 1008 may be disposed at a different position even if the display device is a portable device.

The display device according to the embodiment may be used for a display unit of a photoelectric conversion device, such as an image pickup apparatus including an optical unit including multiple lenses and an image pickup device that receives light passing through the optical unit. The image pickup apparatus may include a display unit that displays information acquired by the image pickup device. The display unit may be a display unit exposed to the outside of the image pickup apparatus or a display unit disposed in a finder. The image pickup apparatus may be a digital camera or a digital camcorder.

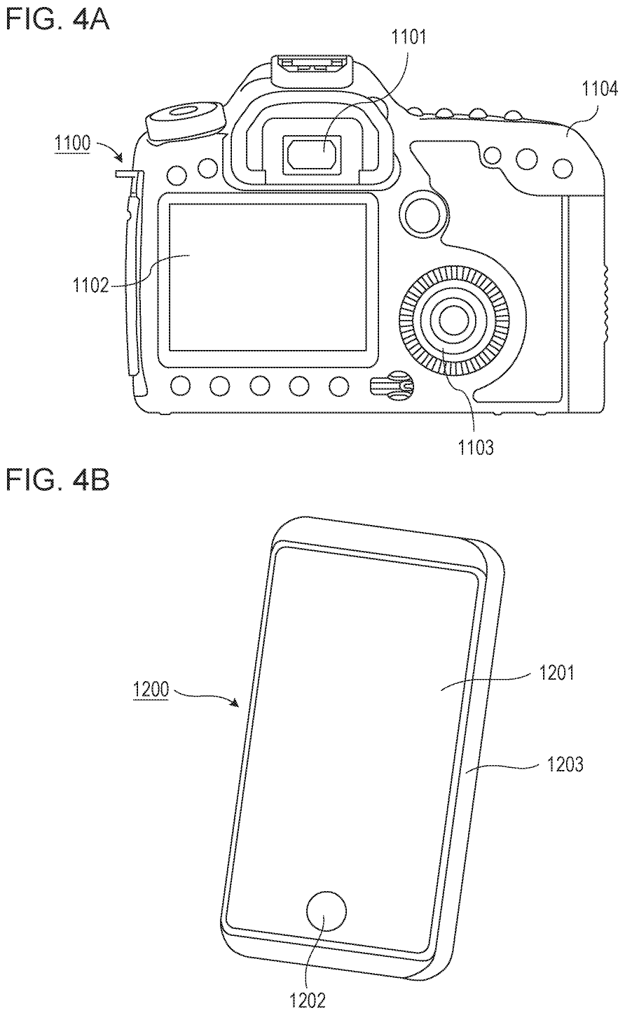

FIG. 4A is a schematic view illustrating an example of an image pickup apparatus according to the embodiment. An image pickup apparatus 1100 may include a viewfinder 1101, a rear display 1102, an operation unit 1103, and a housing 1104. The viewfinder 1101 may include the display device according to the embodiment. In this case, the display device may display environmental information, imaging instructions, and so forth in addition to an image to be captured. The environmental information may include, for example, the intensity of external light, the direction of the external light, the moving speed of a subject, and the possibility that a subject is shielded by a shielding material.

The timing suitable for imaging is only for a short time; thus, the information may be displayed as soon as possible. Accordingly, the display device including the organic light-emitting device according to the embodiment can be used because of its short response time. The display device including the organic light-emitting device can be used more suitably than liquid crystal displays for these units require to have a high display speed.

The image pickup apparatus 1100 includes an optical unit (not illustrated). The optical unit includes multiple lenses and is configured to form an image on an image pickup device in the housing 1104. The relative positions of the multiple lenses can be adjusted to adjust the focal point. This operation can also be performed automatically.

The display device according to the embodiment may include a color filter having red, green, and blue portions. In the color filter, the red, green, and blue portions may be arranged in a delta arrangement.

A display device according to the embodiment may be used for a display unit of an electronic apparatus, such as a portable terminal. In that case, the display device may have both a display function and an operation function. Examples of the portable terminal include mobile phones, such as smartphones, tablets, and head-mounted displays.

FIG. 4B is a schematic view illustrating an example of an electronic apparatus according to the embodiment. An electronic apparatus 1200 includes a display unit 1201, an operation unit 1202, and a housing 1203. The housing 1203 may accommodate a circuit, a printed circuit board including the circuit, a battery, and a communication unit. The operation unit 1202 may be a button or a touch-screen-type reactive unit. The operation unit may be a biometric recognition unit that recognizes a fingerprint to release the lock or the like. An electronic apparatus having a communication unit can also be referred to as a communication apparatus.

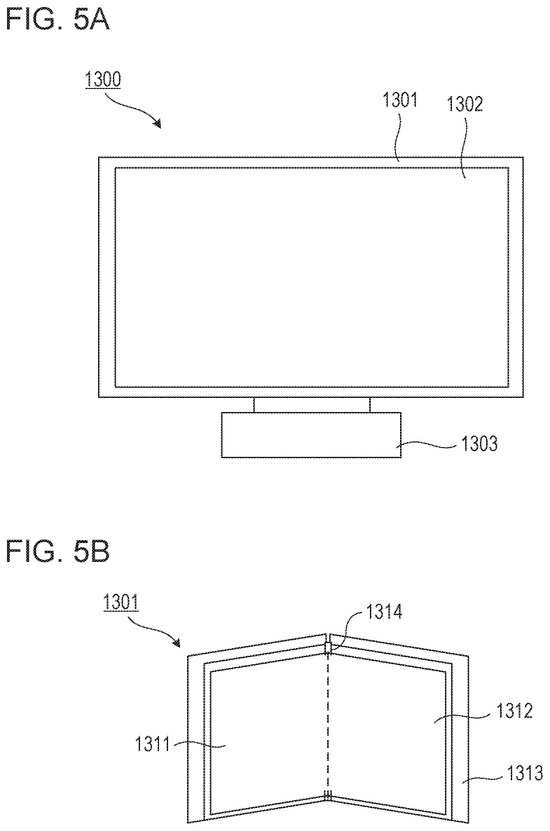

FIGS. 5A and 5B are schematic views illustrating examples of a display device according to the embodiment. FIG. 5A illustrates a display device, such as a television monitor or a personal computer monitor. A display device 1300 includes a frame 1301 and a display unit 1302. The display unit 1302 may include an organic electroluminescent element according to the embodiment. The display device 1300 also includes a base 1303 that supports the frame 1301 and the display unit 1302. The base 1303 is not limited to a form illustrated in FIG. 5A. The lower side of the frame 1301 may also serve as a base. The frame 1301 and the display unit 1302 may be curved and may have a radius of curvature of 5,000 mm or more and 6,000 mm or less.

FIG. 5B is a schematic view illustrating another example of a display device according to the embodiment. A display device 1310 illustrated in FIG. 5B can be folded and is what is called a foldable display device. The display device 1310 includes a first display portion 1311, a second display portion 1312, a housing 1313, and an inflection point 1314. The first display portion 1311 and the second display portion 1312 may include a light-emitting device according to the embodiment. The first display portion 1311 and the second display portion 1312 may be a single, seamless display device. The first display portion 1311 and the second display portion 1312 can be divided from each other at the inflection point. The first display portion 1311 and the second display portion 1312 may display different images from each other. Alternatively, a single image may be displayed in the first and second display portions.

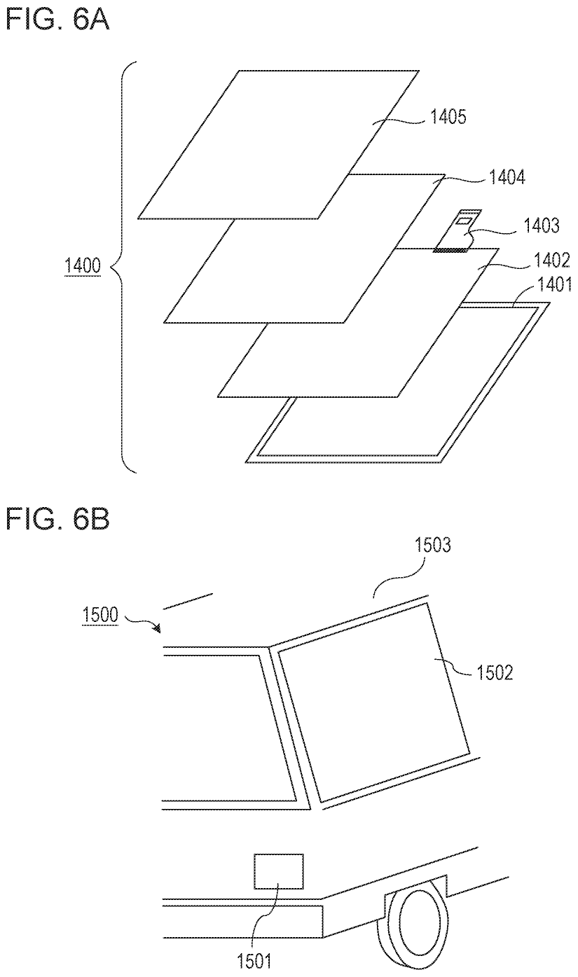

FIG. 6A is a schematic view illustrating an example of a lighting device according to the embodiment. A lighting device 1400 may include a housing 1401, a light source 1402, a circuit board 1403, an optical filter 1404 that transmits light emitted from the light source 1402, and a light diffusion unit 1405. The light source 1402 may include an organic light-emitting device according to the embodiment. The optical filter 1404 may be a filter that improves the color rendering properties of the light source. The light diffusion unit 1405 can effectively diffuse light from the light source to deliver the light to a wide range when used for illumination and so forth. The optical filter 1404 and the light diffusion unit 1405 may be disposed at the light emission side of the lighting device. A cover may be disposed at the outermost portion, as needed.

The lighting device is, for example, a device that lights a room. The lighting device may emit light of white, neutral white, or any color from blue to red. A light control circuit that controls the light may be provided. The lighting device may include the organic light-emitting device according to the embodiment and a power supply circuit coupled thereto. The power supply circuit is a circuit that converts an AC voltage into a DC voltage. The color temperature of white is 4,200 K, and the color temperature of neutral white is 5,000 K. The lighting device may include a color filter.

The lighting device according to the embodiment may include a heat dissipation unit. The heat dissipation unit is configured to release heat in the device to the outside of the device and is composed of, for example, a metal having a high specific heat and liquid silicone.

FIG. 6B is a schematic view illustrating an automobile as an example of a movable body. The automobile includes a tail lamp, which is an example of lighting tools. An automobile 1500 includes a tail lamp 1501 and may be configured to light the tail lamp when a brake operation or the like is performed.

The tail lamp 1501 may include an organic light-emitting device according to the embodiment. The tail lamp 1501 may include a protective member that protects the organic light-emitting device. The protective member may be composed of any transparent material having high strength to some extent and can be composed of, for example, polycarbonate. The polycarbonate may be mixed with, for example, a furandicarboxylic acid derivative or an acrylonitrile derivative.

The automobile 1500 may include an automobile body 1503 and windows 1502 attached thereto. The windows 1502 may be transparent displays if the windows are not used to check the front and back of the automobile. The transparent displays may include an organic light-emitting device according to the embodiment. In this case, the components, such as the electrodes, of the organic light-emitting device are formed of transparent members.

The movable body according to the embodiment may be, for example, a ship, an aircraft, or a drone. The movable body may include a body and a lighting tool attached to the body. The lighting tool may emit light to indicate the position of the body. The lighting tool includes the organic light-emitting device according to the embodiment.

As described above, when the device including the organic light-emitting device according to the embodiment is used, a stable display can be obtained with good image quality even for a long time display.

### EXAMPLES

Examples of the present disclosure will be described below.

### Synthesis Example

The present disclosure will be described below with reference to synthesis examples. However, the present invention is not limited thereto.

### Synthesis Example 1: Synthesis of Exemplified Compound A6

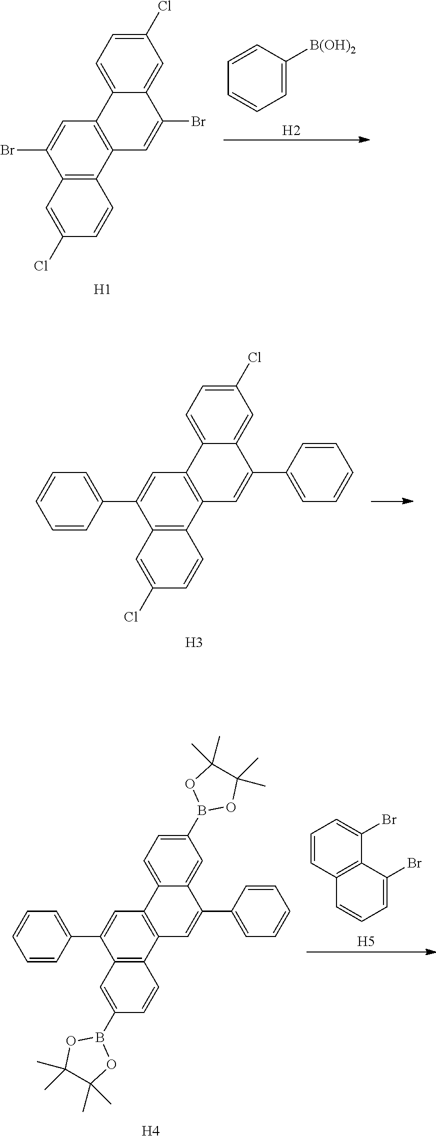

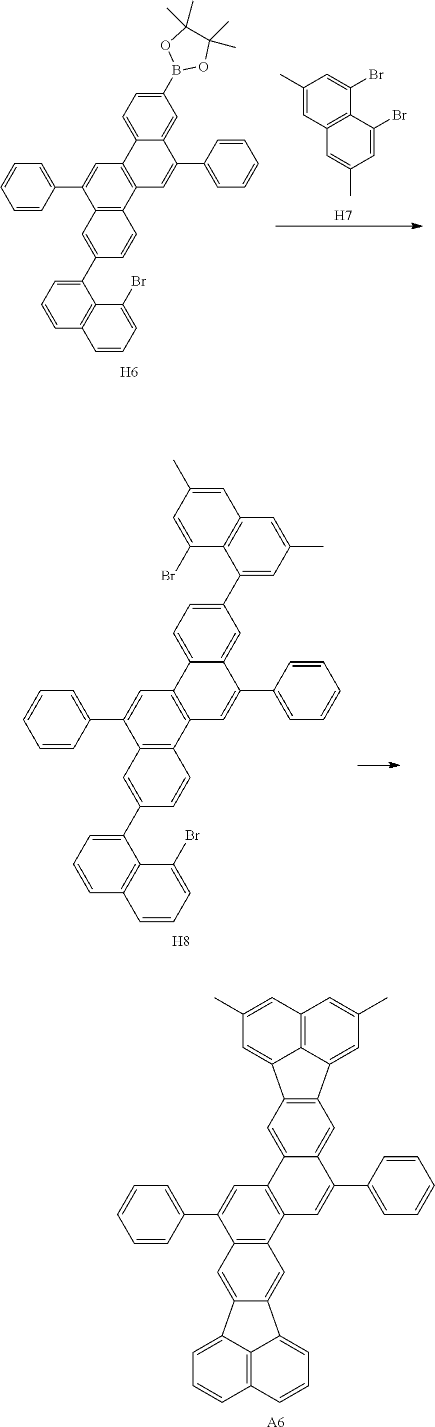

(1) Synthesis of Compound H3

Reagents and solvents described below were charged into a 200-mL recovery flask.

Compound H1: 1.00 g (2.2 mmol)
Compound H2: 0.80 g (6.6 mmol)
Pd(PPh₃)₄: 0.02 g

### Toluene: 100 mL

### Ethanol: 50 mL

2 M Aqueous solution of sodium carbonate: 100 mL

The reaction solution was heated to 80° C. under a stream of nitrogen and stirred at this temperature (80° C.) for 6 hours. After completion of the reaction, water was added thereto, and then liquid-liquid extraction was performed. Dissolution was performed with chloroform. Purification was performed by column chromatography (chloroform), and then recrystallization was performed in chloroform/methanol to give 0.84 g (yield: 85%) of compound H3 as a white solid.

(2) Synthesis of Compound H4

Reagents and a solvent described below were charged into a 200-mL recovery flask.

Compound H3: 0.80 g (1.8 mmol)
Bis(pinacolato)diboron: 1.81 g (7.1 mmol)

### Pd(dba)₂: 0.10 g

### XPhos: 0.26 g

### AcOK: 0.70 g

### Xylene: 80 mL

The reaction solution was heated to 80° C. under a stream of nitrogen and stirred at this temperature (80° C.) for 6 hours. After completion of the reaction, filtration was performed through Celite. The resulting solution was concentrated. The resultant concentrate was dissolved in toluene, purified by column chromatography (toluene), and dispersed and washed with heptane to give 0.91 g (yield: 80%) of compound H4 as a white solid.

(3) Synthesis of Compound H6

Reagents and a solvent described below were charged into a 200-mL recovery flask.

Compound H4: 0.90 g (1.4 mmol)
Compound H5: 0.37 g (1.3 mmol)
Pd(PPh₃)₂Cl₂: 0.01 g

### DMSO: 90 mL

Sodium carbonate: 0.88 g

The reaction solution was heated to 100° C. under a stream of nitrogen and stirred at this temperature (100° C.) for 6 hours. After completion of the reaction, water was added to the mixture to prepare a dispersion, which was filtered. The resultant filtered product was purified by column chromatography (toluene), and dispersed and washed with heptane to give 0.50 g (yield: 50%) of compound H6 as a white solid.

(4) Synthesis of Compound H8

Reagents and a solvent described below were charged into a 200-mL recovery flask.

Compound H6: 0.50 g (0.7 mmol)
Compound H7: 0.33 g (1.1 mmol)
Pd(PPh₃)₂Cl₂: 0.01 g

### DMSO: 50 mL

Sodium carbonate: 0.44 g

The reaction solution was heated to 100° C. under a stream of nitrogen and stirred at this temperature (100° C.) for 6 hours. After completion of the reaction, water was added to the mixture to prepare a dispersion, which was filtered. The resultant filtered product was purified by column chromatography (toluene), and dispersed and washed with heptane to give 0.23 g (yield: 40%) of compound H8 as a white solid.

(5) Synthesis of Exemplified Compound A6

Reagents and a solvent described below were charged into a 20-mL recovery flask.

Compound H8: 0.20 g (0.24 mmol)
Pd(PPh₃)₂Cl₂: 0.02 g

### AcOK: 0.14 g

### DMAc: 10 mL

The reaction solution was heated to 165° C. under a stream of nitrogen and stirred for 6 hours. After completion of the reaction, addition of ethanol to the mixture precipitated crystals, which were filtered. The filtered crystals were dispersed and washed successively with water, ethanol, and heptane. The resultant yellow crystals were dissolved in toluene by heating, hot-filtered, and recrystallized in toluene/methanol to give 0.05 g (yield: 30%) of yellow exemplified compound A6.

The purity of this compound was determined by high-performance liquid chromatography (HPLC) and found to be 99% or more.

Exemplified compound A6 was subjected to mass spectrometry with MALDI-TOF-MS (Autoflex LRF, available from Bruker Corporation).

[MALDI-TOF-MS]

Measured value: m/z=656.88, calculated value: C₅₂H₃₂=656.83

### Synthesis Example 2: Synthesis of Exemplified Compound A19

Exemplified compound A19 was prepared in the same method as Synthesis Example 1, except that compound H9 illustrated below was used instead of compound H3.

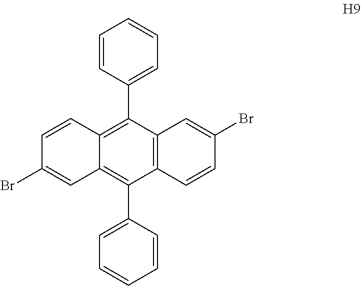 [SMILES: Brc1ccc2c(-c3ccccc3)c3cc(Br)ccc3c(-c3ccccc3)c2c1]

The purity of the resulting compound was evaluated by HPLC and found to be 98% or more.

The compound was subjected to mass spectrometry with MALDI-TOF-MS (Autoflex LRF, available from Bruker Corporation).

[MALDI-TOF-MS]

Measured value: m/z=606.46, calculated value: C₄₈H₃₀=606.77

### EXAMPLES

In these examples, calculations were performed using blue-light-emitting materials as examples. In fact, a device including each of the materials has improved chromaticity because the material has a methyl group at a specific position.

### Examples 1 to 9 and Comparative Examples 1 to 17

The emission wavelengths of exemplified compounds A1 to A9 and comparative compounds E1 to E17 were calculated by the following method.

Method for Calculating Emission Wavelength

For the most stable structure calculated by B3LYP/6-31g*, a transition wavelength from the ground state to an excited state calculated by time-dependent density functional theory (TD-B3LYP/6-31g*) is defined as a calculated wavelength.

The molecular orbital calculation described above was performed using Gaussian 09 (Gaussian 09, Revision C. 01, M. J. Frisch, G. W. Trucks, H. B. Schlegel, G. E. Scuseria, M. A. Robb, J. R. Cheeseman, G. Scalmani, V. Barone, B. Mennucci, G. A. Petersson, H. Nakatsuji, M. Caricato, X. Li, H. P. Hratchian, A. E Izmaylov, J. Bloino, G. Zheng, J. L. Sonnenberg, M. Hada, M. Ehara, K. Toyota, R. Fukuda, J. Hasegawa, M. Ishida, T. Nakajima, Y. Honda, O. Kitao, H. Nakai, T. Vreven, J. A. Montgomery, Jr., J. E. Peralta, F. Ogliaro, M. Bearpark, J. J. Heyd, E. Brothers, K. N. Kudin, V. N. Staroverov, T. Keith, R. Kobayashi, J. Normand, K. Raghavachari, A. Rendell, J. C. Burant, S. S. Iyengar, J. Tomasi, M. Cossi, N. Rega, J. M. Millam, M. Klene, J. E. Knox, J. B. Cross, V. Bakken, C. Adamo, J. Jaramillo, R. Gomperts, R. E. Stratmann, O. Yazyev, A. J. Austin, R. Cammi, C. Pomelli, J. W. Ochterski, R. L. Martin, K. Morokuma, V. G. Zakrzewski, G. A. Voth, P. Salvador, J. J. Dannenberg, S. Dapprich, A. D. Daniels, O. Farkas, J. B. Foresman, J. V. Ortiz, J. Cioslowski, and D. J. Fox, Gaussian, Inc., Wallingford Conn., 2010.), which is currently widely used.

TABLE 2

| | | | Calculated | Reduction in | | Corresponding |
|---|---|---|---|---|---|---|
| | | | wavelength/ | wavelength/ | | comparative |
| Example | | | nm | nm | Effect | example |
| 1 | Compound Al | 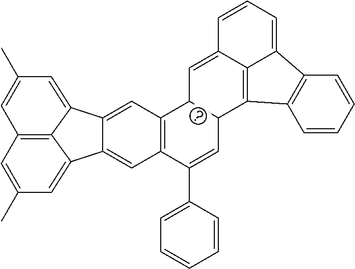 [SMILES: Cc1cc2c3c(cc(C)cc3c1)-c1cc3c(cc1-2)C(c1ccccc1)=CC1C2=c4c(cccc4=CC31)-c1ccccc12] | 466.3 | 1.8 | ◯ | Comparative example 1 |
| 2 | Compound A2 | 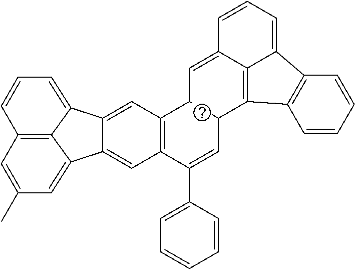 [SMILES: Cc1cc2c3c(cccc3c1)-c1cc3c(cc1-2)C(c1ccccc1)=CC1C2=c4c(cccc4=CC31)-c1ccccc12] | 467.0 | 1.1 | ◯ | Comparative example 1 |
| 3 | Compound A3 | 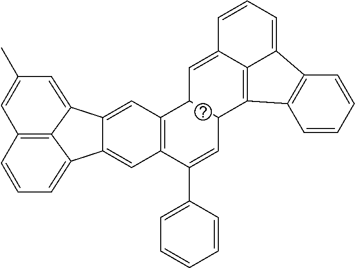 [SMILES: Cc1cc2c3c(cccc3c1)-c1cc3c(cc1-2)C1C=c2cccc4c2=C(c2ccccc2-4)C1C=C3c1ccccc1] | 467.1 | 1.0 | ◯ | Comparative example 1 |
| 4 | Compound A4 | 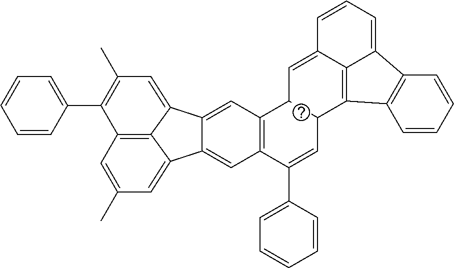 [SMILES: Cc1cc2c3c(cc(C)c(-c4ccccc4)c3c1)-c1cc3c(cc1-2)C(c1ccccc1)=CC1C2=c4c(cccc4=CC31)-c1ccccc12] | 469.5 | 3.7 | ⊙ | Comparative example 2 |
| 5 | Compound A5 | 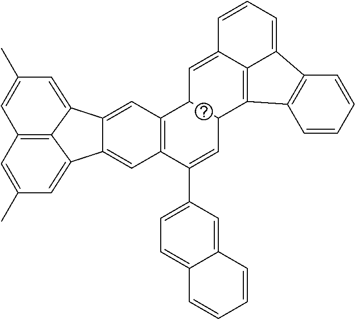 [SMILES: Cc1cc2c3c(cc(C)cc3c1)-c1cc3c(cc1-2)C(c1ccc2ccccc2c1)=CC1C2=c4c(cccc4=CC31)-c1ccccc12] | 466.5 | 1.6 | ◯ | Comparative example 3 |
| 6 | Compound A6 | 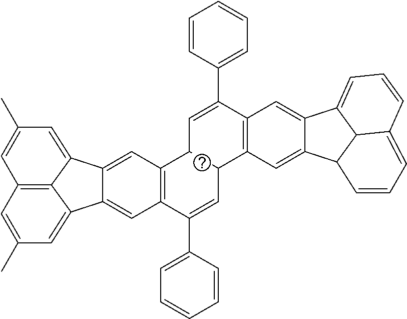 [SMILES: Cc1cc2c3c(cc(C)cc3c1)-c1cc3c(cc1-2)C(c1ccccc1)=CC1c2cc4c(cc2C(c2ccccc2)=CC31)C1=CC=CC2=CC=CC4C21] | 438.9 | 2.2 | ◯ | Comparative example 4 |
| 7 | Compound A7 | 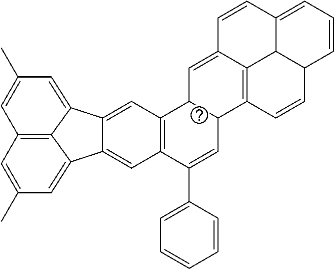 [SMILES: Cc1cc2c3c(cc(C)cc3c1)-c1cc3c(cc1-2)C(c1ccccc1)=CC1C2=C4C(=CC31)C=CC1=CC=CC(C=C2)C14] | 446.7 | 8.1 | ⊙ | Comparative example 5 |
| 8 | Compound A8 | 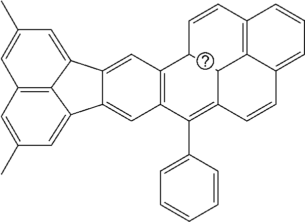 [SMILES: Cc1cc2c3c(cc(C)cc3c1)-c1cc3c(cc1-2)C(c1ccccc1)=C1C=Cc2cccc4c2C1C3C=C4] | 440.8 | 6.6 | ⊙ | Comparative example 6 |
| 9 | Compound A9 | 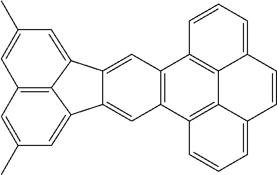 [SMILES: Cc1cc2c3c(cc(C)cc3c1)-c1cc3c(cc1-2)c1cccc2ccc4cccc3c4c21] | 409.2 | 8.9 | ⊙ | Comparative example 12 |

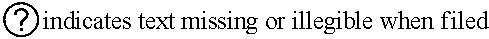

TABLE 3

| | | | Calculated |
|---|---|---|---|
| Comparative example | | | wavelength/nm |
| 1 | Compound E1 | 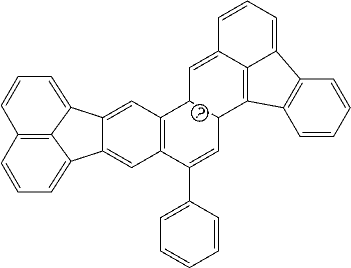 [SMILES: C1=C(c2ccccc2)c2cc3c(cc2C2C=c4cccc5c4=C(c4ccccc4-5)C12)-c1cccc2cccc-3c12] | 468.1 |
| 2 | Compound E2 | 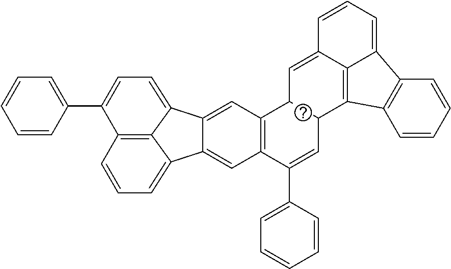 [SMILES: C1=C(c2ccccc2)c2cc3c(cc2C2C=c4cccc5c4=C(c4ccccc4-5)C12)-c1ccc(-c2ccccc2)c2cccc-3c12] | 473.2 |
| 3 | Compound E3 | 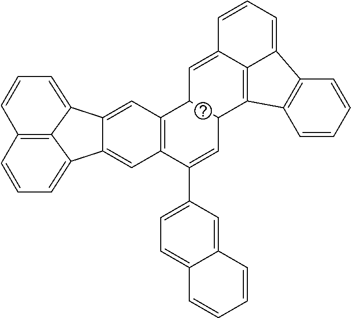 [SMILES: C1=C(c2ccc3ccccc3c2)c2cc3c(cc2C2C=c4cccc5c4=C(c4ccccc4-5)C12)-c1cccc2cccc-3c12] | 468.1 |
| 4 | Compound E4 | 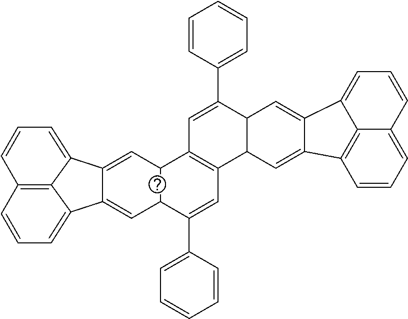 [SMILES: C1=C2C(=CC3C4=C(C=C(c5ccccc5)C13)C1C=C3C(=CC1C(c1ccccc1)=C4)c1cccc4cccc3c14)c1cccc3cccc2c13] | 441.1 |
| 5 | Compound E5 | 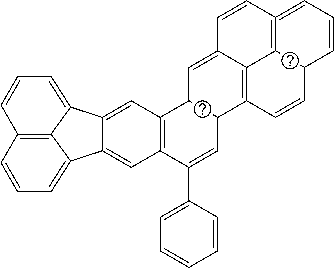 | 454.9 |
| 6 | Compound E6 | 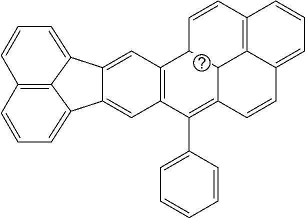 [SMILES: C1=Cc2cccc3c2C2C1=C(c1ccccc1)c1cc4c(cc1C2C=C3)-c1cccc2cccc-4c12] | 447.4 |
| 7 | Compound E7 | 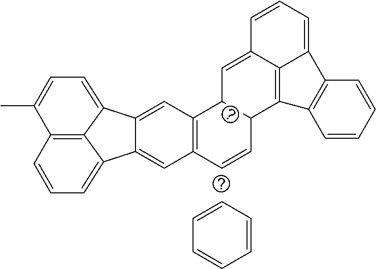 | 470.0 |
| 8 | Compound E8 | 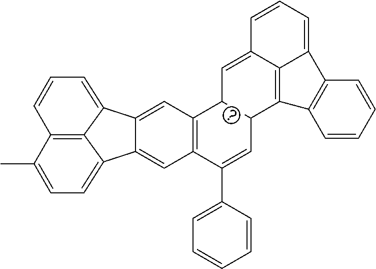 [SMILES: Cc1ccc2c3c(cccc13)-c1cc3c(cc1-2)C(c1ccccc1)=CC1C2=c4c(cccc4=CC31)-c1ccccc12] | 468.0 |
| 9 | Compound E9 | 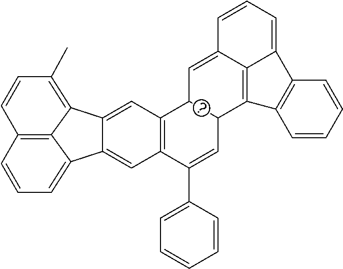 [SMILES: Cc1ccc2cccc3c2c1-c1cc2c(cc1-3)C(c1ccccc1)=CC1C3=c4c(cccc4=CC21)-c1ccccc13] | 469.6 |
| 10 | Compound E10 | 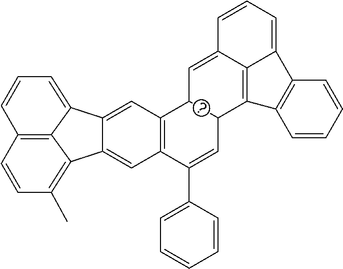 [SMILES: Cc1ccc2cccc3c2c1-c1cc2c(cc1-3)C1C=c3cccc4c3=C(c3ccccc3-4)C1C=C2c1ccccc1] | 468.2 |
| 11 | Compound El1 | 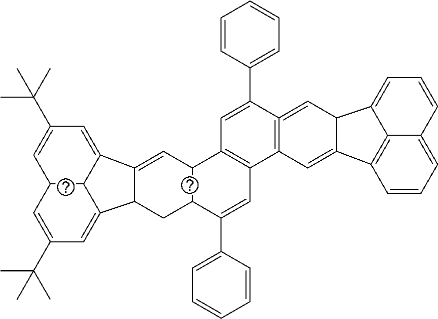 | 438.8 |
| 12 | Compound E12 | 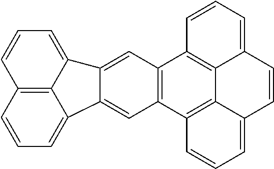 [SMILES: c1cc2c3c(cccc3c1)-c1cc3c(cc1-2)c1cccc2ccc4cccc3c4c21] | 418.1 |
| 13 | Compound E13 | 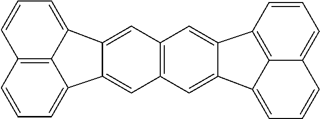 [SMILES: c1cc2cccc3c4cc5cc6c(cc5cc4c(c1)c23)c1cccc2cccc6c21] | 433.2 |
| 14 | Compound E14 | 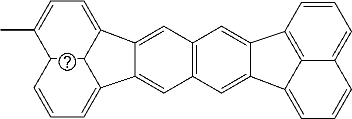 [SMILES: CC1=CC=C2c3cc4cc5c(cc4cc3C3=CC=CC1C32)-c1cccc2cccc-5c12] | 435.7 |
| 15 | Compound E15 | 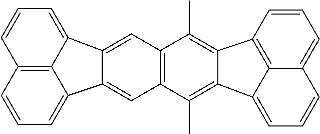 [SMILES: Cc1c2cc3c(cc2c(C)c2c4cccc5cccc(c12)c54)c1cccc2cccc3c21] | 441.7 |
| 16 | Compound E16 | 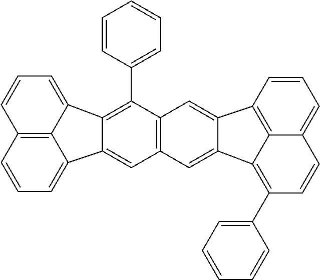 [SMILES: c1ccc(-c2c3cc4c(cc3cc3c5cccc6cccc(c23)c65)c2c(-c3ccccc3)ccc3cccc4c32)cc1] | 445.4 |
| 17 | Compound E17 | 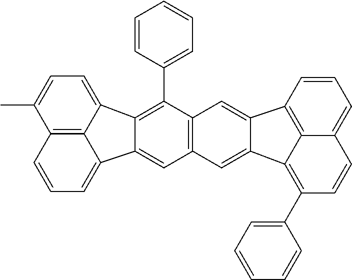 [SMILES: Cc1ccc2c3c(-c4ccccc4)c4cc5c(cc4cc3c3cccc1c32)c1c(-c2ccccc2)ccc2cccc5c21] | 447.9 |

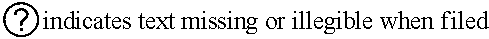

### Examples 1 to 9 and Comparative Examples 1 to 6 and 12

The relationship between each of the examples and a corresponding one of the comparative examples presented in Table 2 is the relationship between a compound in which at least one of R₆ and R₉ is a methyl group and a compound in which the methyl group of the compound is replaced with a hydrogen atom. The effect of the amount of Stokes shift on the emission wavelength calculation shift is included in the calculation of the comparative examples; thus, the difference between the example and the corresponding comparative example indicates the wavelength shift in accordance with the presence or absence of the methyl group. The calculated wavelength in Example 1 is found to be shorter than that in corresponding Comparative example 1. Similarly, the calculated wavelengths in Examples are found to be shorter than those in respective Comparative examples. Regarding the effect of reducing the wavelength, the case of a reduction in wavelength by 0.5 nm or more and less than 3.0 nm was evaluated to be effective (0), and the case of the reduction in wavelength by 3.0 nm or more was evaluated to be highly effective (0). The results of Examples 1 to 3 indicate that two methyl groups are better than one. This demonstrates that the use of the compound in which at least one of R₆ and R₉ is a methyl group enables a reduction in wavelength.

### Comparative Examples 7 to 10

The results of Example 1 and Comparative examples 7 to 10 indicate that the methyl group located at a position other than R₆ or R₉ is ineffective in reducing the wavelength.

### Comparative Example 11

The results of Example 6 and Comparative example 11 indicate that in the case where each of R₆ and R₉ is a tert-butyl group, the effect of reducing the wavelength is provided as in the case of the methyl groups. However, the tert-butyl groups were decomposed during vacuum evaporation; thus, the methyl groups can be used as substituents for reducing the wavelength.

### Comparative Examples 13 to 17

The wavelengths in Comparative examples 14 and 15 in which a methyl group is located at a position other than R₆ or R₉ are longer than that in Comparative example 13 in which no methyl group is present. The wavelength in Comparative example 17 in which a methyl group is located at a position other than R₆ or R₉ is also found to be longer than that in Comparative example 16 in which no methyl group is present.

### Examples 10 to 29 and Comparative Examples 18 to 22 Example 10

In this example, an organic electroluminescent device having a structure presented in Table 4 was produced, the structure being a bottom-emission structure in which an anode, a hole injection layer, a hole transport layer, an electron-blocking layer, a light-emitting layer, a hole-blocking layer, an electron transport layer, an electron injection layer, and a cathode were sequentially formed on a substrate.

An ITO film was formed on a glass substrate and subjected to desired patterning to form an ITO electrode (anode). The ITO electrode had a thickness of 100 nm. The substrate on which the ITO electrode had been formed in this way was used as an ITO substrate in the following steps. Next, vacuum evaporation by resistance heating was performed in a vacuum chamber at a pressure of 1.33×10⁻⁴ Pa to continuously form organic compound layers and electrode layers presented in Table 4 on the ITO substrate. Here, the opposite electrode (metal electrode layer, cathode) had an electrode area of 3 mm².

TABLE 4

| | Material | | | Thickness/nm |
|---|---|---|---|---|
| Cathode | A1 | | | 100 |
| Electron injection | LiF | | | 1 |
| layer (EIL) | | | | |
| Electron transport | ET5 | | | 35 |
| layer (ETL) | | | | |
| Hole-blocking layer | ET17 | | | 10 |
| (HBL) | | | | |
| Emission layer | host | EM3 | ratio by mass | 25 |
| (EML) | guest | A1 | EM3:A1 = 99:1 | |

| | | |
|---|---|---|
| Electron-blocking | HT12 | 45 |
| layer (EBL) | | |
| Hole transport layer | HT3 | 68 |
| (HTL) | | |
| Hole injection layer | HT16 | 5 |
| (HIL) | | |

### Examples 11 to 29 and Comparative Examples 18 to 22

Organic electroluminescent devices were produced as in Example 10, except that guests in the light-emitting layers were changed to compounds presented in Table 5.

The compounds used in Comparative Examples 18 to 22 are described below. The relationship between exemplified compound A1 and comparative compound D1 is the relationship between a compound in which R₆ and R₉ are methyl groups and a compound in which the methyl groups of the compound are replaced with tert-butyl groups. Each of the relationships between exemplified compound A5 and comparative compound D2, between exemplified compound A6 and comparative compound D3, between exemplified compound A7 and comparative compound D4, and between exemplified compound A8 and comparative compound D5 is the same as described above.

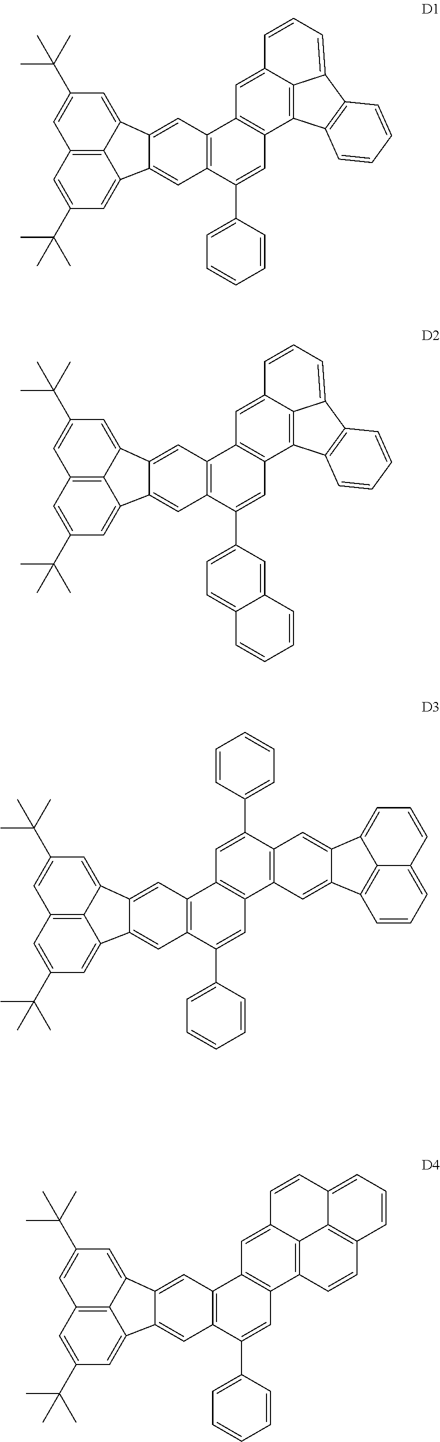

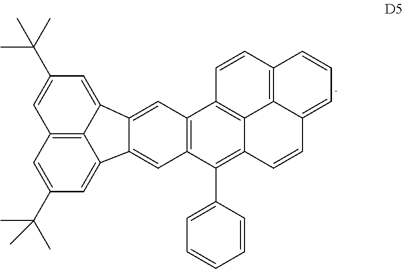 [SMILES: CC(C)(C)c1cc2c3c(cc(C(C)(C)C)cc3c1)-c1cc3c(cc1-2)c(-c1ccccc1)c1ccc2cccc4ccc3c1c24]

TABLE 5

| | | | Improvement in | |
|---|---|---|---|---|
| | EML | | chromaticity | Operating |
| Example No. | Host | Guest | coordinate | half-life |
| Example 10 | EM3 | A1 | ◯ | A |
| Example 11 | EM3 | A2 | ◯ | A |
| Example 12 | EM3 | A3 | ◯ | A |
| Example 13 | EM3 | A4 | ◯ | A |
| Example 14 | EM3 | A5 | ◯ | A |
| Example 15 | EM3 | A6 | ◯ | A |
| Example 16 | EM3 | A7 | ◯ | B |
| Example 17 | EM3 | A8 | ◯ | B |
| Example 18 | EM3 | A9 | ◯ | A |
| Example 19 | EM3 | A10 | ◯ | A |
| Example 20 | EM3 | B1 | ◯ | C |
| Example 21 | EM3 | B2 | ◯ | C |
| Example 22 | EM3 | B3 | ◯ | C |
| Example 23 | EM3 | B4 | ◯ | C |
| Example 24 | EM3 | B5 | ◯ | C |
| Example 25 | EM3 | C1 | ◯ | D |
| Example 26 | EM3 | C2 | ◯ | D |
| Example 27 | EM3 | C3 | ◯ | D |
| Example 28 | EM3 | C4 | ◯ | D |
| Example 29 | EM3 | C5 | ◯ | D |
| Comparative | EM3 | D1 | ◯ | E |
| example 18 | | | | |
| Comparative | EM3 | D2 | ◯ | E |
| example 19 | | | | |
| Comparative | EM3 | D3 | ◯ | E |
| example 20 | | | | |
| Comparative | EM3 | D4 | ◯ | E |
| example 21 | | | | |
| Comparative | EM3 | D5 | ◯ | E |
| example 22 | | | | |

Improvement in Chromaticity Coordinate of Organic Light-Emitting Device

The effect of reducing the wavelength of each of the organic light-emitting devices was evaluated in terms of the chromaticity coordinates of the emission spectrum of each organic light-emitting device. Emission spectra of the produced organic light-emitting devices were measured. The current-voltage characteristics were measured with a 4140B microammeter, available from Hewlett-Packard Company. The luminance and emission spectra were measured with an SR-3A spectroradiometer, available from Topcon Technohouse Corporation. The emission spectra were measured by applying a current of 10 mA/cm².

Table 5 presents the results of comparing the chromaticity of the emission spectra of the organic light-emitting devices with that of the corresponding devices each including the corresponding compound in which R₆ and R₉ are each a hydrogen atom. The compound group used was a compound group that emits blue light; thus, the case where the chromaticity coordinates changed in the direction of increasing the purity of blue-light emission was evaluated to be “effective (0)”. For example, in Example 10, the organic light-emitting device including exemplified compound A1 had improved chromaticity in terms of blue-light emission, compared with an organic light-emitting device (not described) including comparative compound E1.

As a reference value for the target chromaticity of blue-light emission, let us consider CIE (x, y)=(0.15, 0.06) in the sRGB profile. The emission chromaticity of the organic light-emitting device of each Example shifted toward the target chromaticity coordinates (0.15, 0.06), and the chromaticity coordinates were improved. The organic compound according to an embodiment of the present disclosure emitted light having a short wavelength because at least one of R₆ and R₉ is a methyl group; thus, the chromaticity of blue-light emission of the organic light-emitting device was improved.

Operating Life of Organic Light-Emitting Device

The produced organic light-emitting devices were subjected to a continuous operation test at a current density of 100 mA/cm² to measure the half-life at which the luminance was decreased by half. Evaluation criteria were described below. Table 5 presents the results.

A: 180 hours or more
B: 150 hours or more and less than 180 hours
C: 130 hours or more and less than 150 hours
D: 100 hours or more and less than 130 hours
E: less than 100 hours

As presented in Table 5, the half-life in each of Examples 10 to 19 was found to be 150 hours or more (rated as A or B). It was thus found that the compounds that satisfy (A) to (E) described above and that consist only of carbon and hydrogen can have better durability characteristics. Examples 10 to 15, 18, and 19 (rated as A) were superior to Examples 16 and 17 (rated as B). This is probably because the presence of two five-membered carbon ring structures increases the ionization potential of the molecule to improve the oxidation stability. This is also supported by the results of the calculated value of HOMO (HOMO (calc.)) in Table 6. The calculated value of HOMO was determined by calculating the most stable structure in the ground state with B3LYP/6-31g*. In Table 6, a larger negative HOMO value indicates a higher ionization potential.

TABLE 6

| Compound | HOMO(calc.)/eV | Number of five-membered ring |
|---|---|---|
| Compound A1 | −5.2 | 2 |
| Compound A2 | −5.2 | 2 |
| Compound A3 | −5.2 | 2 |
| Compound A4 | −5.2 | 2 |
| Compound A5 | −5.2 | 2 |
| Compound A6 | −5.1 | 2 |
| Compound A7 | −5.0 | 1 |
| Compound A8 | −4.9 | 1 |

The half-life in each of Examples 20 to 24 was found to be 130 hours or more (rated as C). The half-life in each of Examples 25 to 29 was found to be 100 hours or more (rated as D). It was thus found that the compounds that satisfy (A), (B), and (C1) to (E1) and in which the bonds between the basic skeletons and the substituents are carbon-carbon bonds can have good durability characteristics.

The effect of reducing the wavelength is provided in Comparative examples 18 to 22. However, the half-life did not reach 100 hours at the longest (rated as E). The reason for this is presumably that from the fact that the degree of vacuum was deteriorated during the vacuum deposition of the light-emitting layer of the organic light-emitting device of each of Comparative examples 18 to 22, the film containing the decomposition products of the material was formed during film formation to decrease the operating life. Accordingly, it was found that the methyl group can be used rather than the tert-butyl group.

From the results of Examples 10 to 29 described above, it is possible to provide the organic light emitting devices having improved chromaticity of blue-light emission and excellent operating lives.

### Example 30

In this example, an organic electroluminescent device having a structure presented in Table 7 was produced, the structure being a top-emission structure in which an anode, a hole injection layer, a hole transport layer, an electron-blocking layer, a first light-emitting layer, a second light-emitting layer, a hole-blocking layer, an electron transport layer, an electron injection layer, and a cathode were sequentially formed on a substrate.

A Ti film having a thickness of 40 nm was formed on a glass substrate by a sputtering method and subjected to patterning by photolithography to form the anode. Here, the opposite electrode (metal electrode layer, cathode) had an electrode area of 3 mm². Subsequently, materials and the substrate including the cleaned electrode were attached to a vacuum evaporation apparatus (available from Ulvac, Inc.), which was evacuated to 1.33×10⁻⁴ Pa (1×10⁻⁶ Torr) and subjected to UV/ozone cleaning. Then layers were formed so as to achieve the layer configuration described in Table 7. Finally, sealing was performed in a nitrogen atmosphere.

TABLE 7

| | | | | Thickness |
|---|---|---|---|---|
| | Material | | | (nm) |
| Cathode | Mg | ratio by mass | | 10 |
| | Ag | Mg:Ag = 50:50 | | |
| Electron | LiF | | | 1 |
| injection layer | | | | |
| (EIL) | | | | |
| Electron | ET2 | | | 30 |
| transport layer | | | | |
| (ETL) | | | | |
| Hole-blocking | ET12 | | | 70 |
| layer (HBL) | | | | |
| Second light- | second host | EM1 | ratio by mass | 10 |
| emitting layer | second guest | A1 | EM1:A1 = 99.4:0.6 | |
| (2nd EML) | (blue dopant) | | | |
| First light- | first host | EM1 | ratio by mass | 10 |
| emitting layer | first guest (red | RD7 | EM1:RD7:GD9 = | |
| (1st EML) | dopant) | | 96.7:0.3:3.0 | |
| | third guest | GD9 | | |
| | (green dopant) | | | |

| | | |
|---|---|---|
| Electron- | HT7 | 10 |
| blocking layer | | |
| (EBL) | | |
| Hole transport | HT2 | 20 |
| layer (HTL) | | |
| Hole injection | HT16 | 5 |
| layer (HIL) | | |

The characteristics of the produced device were measured and evaluated. The device exhibited good white-light emission. From the white emission spectrum, the chromaticity coordinates of blue after passing through an RGB color filter were estimated, and the chromaticity coordinates of blue in the sRGB color space were (0.15, 0.12).

### Examples 31 and 32 and Comparative Examples 23 to 26

Organic light-emitting devices were produced in the same way as in Example 30, except that the materials were changed to compounds presented in Table 8 as appropriate. The characteristics of the produced devices were measured and evaluated as in Example 30. Table 8 presents the measurement results. Evaluation criteria for the operation half-life were as follows:

◯: 150 hours or more; and
x: less than 150 hours.

TABLE 8

| | | | | | | Blue | |
|---|---|---|---|---|---|---|---|
| | 1st EML | | | 2nd EML | | chromaticity | |
| | First | First | Third | Second | Second | coordinates | Operating |
| | host | guest | guest | host | guest | (x, y) | half-life |
| Example 31 | EM1 | RD7 | GD9 | EM1 | A1 | (0.15, 0.12) | ∘ |
| Comparative | EM1 | RD7 | GD9 | EM1 | E1 | (0.15, 0.16) | ∘ |
| example 23 | | | | | | | |
| Example 32 | EM1 | RD7 | GD9 | EM1 | A6 | (0.15, 0.07) | ∘ |
| Comparative | EM1 | RD7 | GD9 | EM1 | E4 | (0.15, 0.10) | ∘ |
| example 24 | | | | | | | |
| Comparative | EM1 | RD7 | GD9 | EM1 | D1 | (0.15, 0.12) | x |
| example 25 | | | | | | | |
| Comparative | EM1 | RD7 | GD9 | EM1 | D3 | (0.15, 0.07) | x |
| example 26 | | | | | | | |

As presented in Table 8, in Example 31, the blue chromaticity coordinates were improved so as to be brought close to pure blue, compared with Comparative example 23. In Example 32, the blue chromaticity coordinates were improved so as to be brought close to pure blue, compared with Comparative example 24. The relationships between each of the examples and a corresponding one of the comparative examples is the relationship between a compound in which R₆ and R₉ are methyl groups and a compound in which the methyl groups of the compound are replaced with hydrogen atoms.

The operating life of the organic light-emitting device of Example 31 was about twice as long as that of Comparative example 25. Similarly, the operating life of the organic light-emitting device of Example 32 was about twice as long as that of Comparative example 26. The relationship between each of the examples and a corresponding one of the comparative examples is the relationship between a compound in which R₆ and R₉ are methyl groups and a compound in which the methyl groups of the compound are replaced with tert-butyl groups.

From the results described above, in the case of performing evaluation in the form of the white-light-emitting devices, the white-light-emitting devices according to embodiments of the present disclosure tend to expand the color gamut with respect to the sRGB color gamut in a blue emission region. This is because the compounds according to embodiments of the present disclosure emit blue light at shorter wavelengths.

According to an embodiment of the present disclosure, it is possible to provide an organic compound having a shorter emission wavelength and high sublimation properties.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2019-143120, filed Aug. 2, 2019, which is hereby incorporated by reference herein in its entirety.

What is claimed is:

1. An organic compound represented by formula [1]: 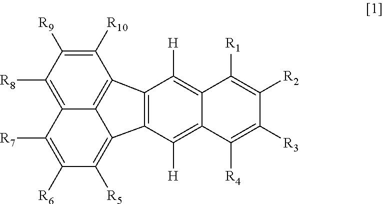
wherein in formula [1], R₁ to R₁₀ are each independently selected from the group consisting of a hydrogen atom, a substituted or unsubstituted alkyl group, a substituted or unsubstituted alkoxy group, a substituted or unsubstituted aryl group, a substituted or unsubstituted heterocyclic group, and a substituted or unsubstituted amino group, provided that at least one of R₆ and R₉ is a methyl group, and sets of R₁ and R₂, R₂ and R₃, and R₃ and R₄ are each independently optionally taken together to form a ring.

2. The organic compound according to claim 1, wherein the aryl group has 6 or more and 18 or less carbon atoms.

3. The organic compound according to claim 1, wherein at least one of the sets of R₁ and R₂, R₂ and R₃, and R₃ and R₄ is taken together to form a ring.

4. The organic compound according to claim 1, wherein the ring formed by taking at least one of the sets of R₁ and R₂, R₂ and R₃, and R₃ and R₄ together is an aromatic ring.

5. The organic compound according to claim 1, wherein the ring formed by taking at least one of the sets of R₁ and R₂, R₂ and R₃, and R₃ and R₄ together is a condensed polycyclic aromatic ring including a five-membered carbon ring.

6. The organic compound according to claim 1, wherein R₅, R₇, R₈, R₁₀, and a group that does not form a ring out of R₁ to R₄ are each a hydrogen atom, an aryl group, or a heterocyclic group bonded through a carbon atom.

7. The organic compound according to claim 1, wherein the ring formed by taking at least one of the sets of R₁ and R₂, R₂ and R₃, and R₃ and R₄ together optionally includes a substituent, and
wherein the substituent is an aryl group or a heterocyclic group bonded through a carbon atom.

8. The organic compound according to claim 1, wherein R₆ and R₉ are each a methyl group.

9. The organic compound according to claim 1, wherein one of R₆ and R₉ is a methyl group, and the other is a hydrogen atom.

10. An organic light-emitting device, comprising:
a first electrode;
a second electrode; and
an organic compound layer disposed between the first electrode and the second electrode,
the organic compound layer including at least one layer containing the organic compound according to claim 1.

11. The organic light-emitting device according to claim 10, wherein the at least one layer containing the organic compound is a light-emitting layer.

12. The organic light-emitting device according to claim 11, wherein the organic light-emitting device emits blue light.

13. The organic light-emitting device according to claim 12, further comprising:
another light-emitting layer stacked on the light-emitting layer,
wherein the another light-emitting layer emits light of a color different from a color of light emitted from the light-emitting layer.

14. The organic light-emitting device according to claim 13, wherein the organic light-emitting device emits white light.

15. A display device, comprising:
multiple pixels,
wherein at least one of the multiple pixels includes the organic light-emitting device according to claim 10 and a transistor coupled to the organic light-emitting device.

16. A photoelectric conversion device, comprising:
an optical unit including multiple lenses;
an image pickup device that receives light passing through the optical unit; and
a display unit that displays an image captured by the image pickup device, the display unit including the organic light-emitting device according to claim 10.

17. An electronic apparatus, comprising:
a display unit including the organic light-emitting device according to claim 10;
a housing provided with the display unit; and
a communication unit disposed in the housing, the communication unit being configured to communicate with an outside.

18. A lighting device, comprising:
a light source including the organic light-emitting device according to claim 10; and
a light diffusion unit or an optical filter that transmits light emitted from the light source.

19. A movable body, comprising:
a lighting tool including the organic light-emitting device according to claim 10; and
a body provided with the lighting tool.
